(19) United States
(12) Patent Application Publication
(10) Pub. No.: US 2021/0260055 A1
(43) Pub. Date: Aug. 26, 2021

(54) TOPICAL FORMULATIONS OF DGAT1 INHIBITORS AND THEIR METHODS OF USE

(71) Applicant: Dermavant Sciences GmbH, Basel (CH)

(72) Inventors: Haripriya KALLURI, Morrisville, NC (US); Robert LEMING, Morganton, NC (US); Piyush JAIN, Durham, NC (US)

(21) Appl. No.: 17/253,373

(22) PCT Filed: Jun. 21, 2019
(86) PCT No.: PCT/US2019/038535

§ 371 Date: Dec. 17, 2020

Related U.S. Application Data
(60) Provisional application No. 62/695,441, filed on Jul. 9, 2018.
(60) Provisional application No. 62/688,180, filed on Jun. 21, 2018.

(51) Int. Cl. A61K 31/4965; A61K 9/00; A61K 47/10; A61K 47/38; A61K 47/32

(52) U.S. Cl. CPC A61K 31/4965; A61K 9/0014; A61K 47/32; A61K 47/38; A61K 47/10

(57) ABSTRACT

Embodiments herein are directed to topical compositions comprising 2-(4-(4-(6-carbamoyl-3,5-dimethylpyrazin-2-yl)phenyl)cyclohexyl)acetic acid and a combination of one or more of the following components: benzyl alcohol, PEG 400, diisopropyl adipate, diethyl sebacate, dimethyl isosorbide, propylene glycol, 2-(2-ethoxyethoxy)ethanol, hexylene glycol, oleic acid, polysorbate 80, isopropyl myristate, glycerin, DMSO, water, butylated hydroxytoluene, phenoxyethanol, cellulose polymer, Carbomer Homopolymer Type B, and optionally one or more pharmaceutically acceptable topical excipients. The topical compositions may be used to treat a variety of skin conditions.

### CROSS REFERENCE TO RELATED APPLICATION

The present application claims benefit of and priority to U.S. Provisional No. 62/688,180 entitled “TOPICAL FORMULATIONS OF DGAT1 INHIBITORS AND THEIR METHODS OF USE” filed Jun. 21, 2018 and to U.S. Provisional No. 62/695,441 entitled “TOPICAL FORMULATIONS OF DGAT1 INHIBITORS AND THEIR METHODS OF USE” filed Jul. 9, 2018, the contents of which is hereby incorporated by reference in its entirety.

### SUMMARY OF THE INVENTION

Embodiments herein are directed to a topical composition comprising 2-(4-(4-(6-carbamoyl-3,5-dimethylpyrazin-2-yl)phenyl)cyclohexyl)acetic acid, benzyl alcohol, diisopropyl adipate, diethyl sebacate, dimethyl isosorbide, 2-(2-ethoxyethoxy)ethanol, hexylene glycol, polysorbate 80, and optionally one or more pharmaceutically acceptable topical excipients such as but not limited to PEG 400, ethanol, isopropanol.

Embodiments herein are directed to a topical composition comprising 2-(4-(4-(6-carbamoyl-3,5-dimethylpyrazin-2-yl)phenyl)cyclohexyl)acetic acid, benzyl alcohol, PEG 400, 2-(2-ethoxyethoxy)ethanol, polysorbate 80, and optionally one or more pharmaceutically acceptable topical excipients. In some embodiments, the topical composition further comprises diisopropyl adipate. In some embodiments, the topical composition further comprises diethyl sebacate. In some embodiments, the topical composition further comprises dimethyl isosorbide. In some embodiments, the topical composition further comprises hexylene glycol.

Embodiments herein are directed to a topical composition comprising 2-(4-(4-(6-carbamoyl-3,5-dimethylpyrazin-2-yl)phenyl)cyclohexyl)acetic acid, PEG 400, dimethyl isosorbide, 2-(2-ethoxyethoxy)ethanol, DMSO, and optionally one or more pharmaceutically acceptable topical excipients.

Embodiments herein are directed to a topical composition comprising 2-(4-(4-(6-carbamoyl-3,5-dimethylpyrazin-2-yl)phenyl)cyclohexyl)acetic acid, propylene glycol, oleic acid, polysorbate 80, isopropyl myristate, phenoxyethanol, and optionally one or more pharmaceutically acceptable topical excipients.

Embodiments herein are directed to a topical composition comprising 2-(4-(4-(6-carbamoyl-3,5-dimethylpyrazin-2-yl)phenyl)cyclohexyl)acetic acid, benzyl alcohol, glycerin, and optionally one or more pharmaceutically acceptable topical excipients.

Embodiments herein are directed to a topical composition comprising 2-(4-(4-(6-carbamoyl-3,5-dimethylpyrazin-2-yl)phenyl)cyclohexyl)acetic acid, DMSO, water, and optionally one or more pharmaceutically acceptable topical excipients.

Embodiments herein are directed to a topical composition comprising 2-(4-(4-(6-carbamoyl-3,5-dimethylpyrazin-2-yl)phenyl)cyclohexyl)acetic acid, propylene glycol, water, and optionally one or more pharmaceutically acceptable topical excipients.

Embodiments herein are directed to a topical composition comprising 2-(4-(4-(6-carbamoyl-3,5-dimethylpyrazin-2-yl)phenyl)cyclohexyl)acetic acid, propylene glycol, glycerin, and optionally one or more pharmaceutically acceptable topical excipients.

Embodiments herein are directed to a topical composition comprising 2-(4-(4-(6-carbamoyl-3,5-dimethylpyrazin-2-yl)phenyl)cyclohexyl)acetic acid, propylene glycol, 2-(2-ethoxyethoxy)ethanol, PEG400, butylated hydroxytoluene, and optionally one or more pharmaceutically acceptable topical excipients. In some embodiments, the topical composition further comprises glycerin. In some embodiments, the topical composition further comprises cellulose polymers. In some embodiments, the topical composition further comprises Carbomer Homopolymer Types A, B or C. In some embodiments, the topical composition further comprises Poloxamer.

Embodiments herein are directed to a topical composition comprising 2-(4-(4-(6-carbamoyl-3,5-dimethylpyrazin-2-yl)phenyl)cyclohexyl)acetic acid, 2-(2-ethoxyethoxy)ethanol, PEG400, glycerin, cellulose polymers, butylated hydroxytoluene, and optionally one or more pharmaceutically acceptable topical excipients.

Embodiments herein are directed to a topical composition comprising 2-(4-(4-(6-carbamoyl-3,5-dimethylpyrazin-2-yl)phenyl)cyclohexyl)acetic acid, propylene glycol, 2-(2-ethoxyethoxy)ethanol, glycerin, dimethyl isosorbide, butylated hydroxytoluene, and optionally one or more pharmaceutically acceptable topical excipients. In some embodiments, the topical composition further comprises cellulose polymers. In some embodiments, the topical composition further comprises Carbomer Homopolymer Types A, B or C.

Some embodiments herein are directed to a method of treating skin conditions in a patient in need thereof comprising topically applying a topical composition comprising a therapeutically effective amount of 2-(4-(4-(6-carbamoyl-3,5-dimethylpyrazin-2-yl)phenyl)cyclohexyl)acetic acid, benzyl alcohol, diisopropyl adipate, diethyl sebacate, dimethyl isosorbide, 2-(2-ethoxyethoxy)ethanol, hexylene glycol, polysorbate 80, and optionally one or more pharmaceutically acceptable topical excipients such as but not limited to PEG 400, ethanol, isopropanol.

Some embodiments herein are directed to a method of treating skin conditions in a patient in need thereof comprising topically applying a topical composition comprising 2-(4-(4-(6-carbamoyl-3,5-dimethylpyrazin-2-yl)phenyl)cyclohexyl)acetic acid, benzyl alcohol, PEG 400, 2-(2-ethoxyethoxy)ethanol, polysorbate 80, and optionally one or more pharmaceutically acceptable topical excipients.

Some embodiments herein are directed to a method of treating skin conditions in a patient in need thereof comprising topically applying a topical composition comprising 2-(4-(4-(6-carbamoyl-3,5-dimethylpyrazin-2-yl)phenyl)cyclohexyl)acetic acid, PEG 400, dimethyl isosorbide, 2-(2-ethoxyethoxy)ethanol, DMSO, and optionally one or more pharmaceutically acceptable topical excipients.

Some embodiments herein are directed to a method of treating skin conditions in a patient in need thereof comprising topically applying a topical composition comprising 2-(4-(4-(6-carbamoyl-3,5-dimethylpyrazin-2-yl)phenyl)cyclohexyl)acetic acid, propylene glycol, oleic acid, polysorbate 80, isopropyl myristate, phenoxyethanol, and optionally one or more pharmaceutically acceptable topical excipients.

Some embodiments herein are directed to a method of treating skin conditions in a patient in need thereof comprising topically applying a topical composition comprising 2-(4-(4-(6-carbamoyl-3,5-dimethylpyrazin-2-yl)phenyl)cyclohexyl)acetic acid, benzyl alcohol, glycerin, and optionally one or more pharmaceutically acceptable topical excipients.

Some embodiments herein are directed to a method of treating skin conditions in a patient in need thereof comprising topically applying a topical composition comprising 2-(4-(4-(6-carbamoyl-3,5-dimethylpyrazin-2-yl)phenyl)cyclohexyl)acetic acid, DMSO, water, and optionally one or more pharmaceutically acceptable topical excipients.

Some embodiments herein are directed to a method of treating skin conditions in a patient in need thereof comprising topically applying a topical composition comprising 2-(4-(4-(6-carbamoyl-3,5-dimethylpyrazin-2-yl)phenyl)cyclohexyl)acetic acid, propylene glycol, water, and optionally one or more pharmaceutically acceptable topical excipients.

Some embodiments herein are directed to a method of treating skin conditions in a patient in need thereof comprising topically applying a topical composition comprising 2-(4-(4-(6-carbamoyl-3,5-dimethylpyrazin-2-yl)phenyl)cyclohexyl)acetic acid, propylene glycol, glycerin, and optionally one or more pharmaceutically acceptable topical excipients.

Some embodiments herein are directed to a method of treating skin conditions in a patient in need thereof comprising topically applying a topical composition comprising 2-(4-(4-(6-carbamoyl-3,5-dimethylpyrazin-2-yl)phenyl)cyclohexyl)acetic acid, propylene glycol, 2-(2-ethoxyethoxy)ethanol, PEG400, butylated hydroxytoluene, and optionally one or more pharmaceutically acceptable topical excipients. In some embodiments, the topical composition further comprises cellulose polymers. In some embodiments, the topical composition further comprises Carbomer Homopolymer Types A, B or C. In some embodiments, the topical composition further comprises Poloxamer.

Some embodiments herein are directed to a method of treating skin conditions in a patient in need thereof comprising topically applying a topical composition comprising 2-(4-(4-(6-carbamoyl-3,5-dimethylpyrazin-2-yl)phenyl)cyclohexyl)acetic acid, 2-(2-ethoxyethoxy)ethanol, PEG400, glycerin, cellulose polymer, butylated hydroxytoluene, and optionally one or more pharmaceutically acceptable topical excipients.

Some embodiments herein are directed to a method of treating skin conditions in a patient in need thereof comprising topically applying a topical composition comprising 2-(4-(4-(6-carbamoyl-3,5-dimethylpyrazin-2-yl)phenyl)cyclohexyl)acetic acid, propylene glycol, 2-(2-ethoxyethoxy)ethanol, glycerin, dimethyl isosorbide, butylated hydroxytoluene, and optionally one or more pharmaceutically acceptable topical excipients. In some embodiments, the topical composition further comprises cellulose polymers. In some embodiments, the topical composition further comprises Carbomer Homopolymer Types A, B or C.

### DESCRIPTION OF THE DRAWINGS

For a fuller understanding of the nature and advantages of the present invention, reference should be had to the following detailed description taken in connection with the accompanying drawings, in which:

FIG. 1A provides cell viability data measured by CTB at day 3 of treatment.

FIG. 1B provides cell viability data measured by CTB at day 5 of treatment.

FIG. 2A provides neutral/total lipid ratio data measured at day 3 of treatment. FIG. 2B provides neutral/total lipid ratio data measured at day 5 of treatment.

FIG. 3A provides total lipid normalized to CTB signal at day 3 of treatment.

FIG. 3B provides total lipid normalized to CTB signal at day 5 of treatment.

FIG. 4A provides neutral lipid normalized to CTB signal at day 3 of treatment. FIG. 4B provides neutral lipid normalized to CTB signal at day 5 of treatment.

FIG. 5A provides percent of control (neutral/total) at day 3 of treatment.

FIG. 5B provides percent of control (neutral/total) at day 5 of treatment.

FIG. 6A provides the DMVT-503 dose response curve as a percent of control on day 3 of treatment. FIG. 6B provides the DMVT-503 dose response curve as a percent of control on day 5 of treatment.

FIG. 7 provides cell viability data measured by CTB.

FIG. 8 provides the raw CPM data.

FIG. 9 provides CPM data normalized to CTB signal.

FIG. 10 provides the percent of positive control (LXR+INS).

FIG. 11 provides the DMVT-503 dose response curve as a percent of positive control (LXR+INS).

FIG. 12 provides Caspase 3 data after 48 hour treatment.

FIG. 13 provides Caspase 3 data after 72 hour treatment.

FIG. 14 provides Caspase 3 data after 5 day treatment.

FIG. 15 provides in vitro skin penetration data for drug levels delivered to the epidermis and dermis tissues.

FIG. 16 provides skin permeation results of 6 formulations.

FIG. 17 provides skin penetration results for 6 formulations.

### DETAILED DESCRIPTION

This invention is not limited to the particular processes, compositions, or methodologies described, as these may vary. The terminology used in the description is for the purpose of describing the particular versions or embodiments only, and is not intended to limit the scope of the present invention. Unless defined otherwise, all technical and scientific terms used herein have the same meanings as commonly understood by one of ordinary skill in the art. All publications mentioned herein are incorporated by reference in their entirety. Nothing herein is to be construed as an admission that the invention is not entitled to antedate such disclosure by virtue of prior invention.

It must be noted that, as used herein, and in the appended claims, the singular forms “a,” “an,” and “the” include plural reference unless the context clearly dictates otherwise.

As used herein, the term “about” means plus or minus 15% of the numerical value of the number with which it is being used. Therefore, about 50.0% means in the range of 2.5% to 67.5%.

The term “acne” includes acne vulgaris and cystic acne and is characterized by areas of skin with seborrhea, comedones (blackheads and whiteheads), papules (pinheads), nodules (large papules) and pimples. Acne vulgaris is a disorder resulting from the action of hormones on the skin's oil glands (sebaceous glands), wherein the sebaceous glands of the skin produce excess sebum, and become enlarged and/or infected when the pore opening becomes plugged with a comedone (a mixture of keratin and sebum). The symptoms of acne include plugged pores and outbreaks of inflamed lesions commonly called pimples.

The use of “API” is used to refer to the active pharmaceutical ingredient. Throughout the disclosure API is 2-((1R,4R)-4-(4-(6-carbamoyl-3,5-dimethylpyrazin-2-yl)phenyl)cyclohexyl)acetic acid or 2-(4-(4-(6-carbamoyl-3,5-dimethylpyrazin-2-yl)phenyl)cyclohexyl)acetic acid.

“Administering” when used in conjunction with a composition means to administer a composition to a patient whereby it positively impacts the tissue to which it is targeted, e.g. the skin. “Administering” a composition may be accomplished by, for example, topical administration, or in combination with other known techniques. Administering may be self-administration, wherein the subject in need of such treatment administers a composition or administering may be by a medical or other health care professional or a caretaker of the subject in need of such treatment.

The term “animal,” “patient,” or “subject” as used herein includes, but is not limited to, humans and non-human vertebrates such as wild, domestic and farm animals.

The terms “AZD7687” or “RVT-503” or “DMVT-503” refer to the chemical compound 2-((1R,4R)-4-(4-(6-carbamoyl-3,5-dimethylpyrazin-2-yl)phenyl)cyclohexyl)acetic acid or 2-(4-(4-(6-carbamoyl-3,5-dimethylpyrazin-2-yl)phenyl)cyclohexyl)acetic acid which is a DGAT1 inhibitor, having the chemical structure of formula I:

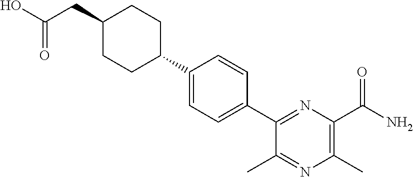 [SMILES: Cc1nc(C)c(-c2ccc([C@H]3CC[C@H](CC(=O)O)CC3)cc2)nc1C(N)=O]

When used herein, the term “Carbomer Homopolymer Types A, B and C” are the three categories of carbomers included in the USP/NF monograph. This term includes numerous trademark names for a polymer of carboxypolymethylene.

As used herein, the terms “comprising,” “comprise,” “comprises,” and “comprised” are inclusive or open-ended and do not exclude additional, unrecited elements or method steps.

As used herein, the term “consists of” or “consisting of” means that the composition or method includes only the elements, steps, or ingredients specifically recited in the particular embodiment or claim.

As used herein, the term “consisting essentially of” or “consists essentially of” means that the composition or method includes only the specified materials or steps and those that do not materially affect the basic and novel characteristics of the claimed invention.

Specific embodiments disclosed herein may be further limited in the claims using “consisting of” or “consisting essentially of” language, rather than “comprising”. In other words, though embodiments described herein use the phrase “comprising” or “comprises,” any embodiment described herein can be replaced with “consisting of”/“consists of” or “consisting essentially of”/“consists essentially of.”

As used herein, the term “HPC” refers to hydroxypropyl cellulose.

The term “therapeutically effective amount” refers to an amount of a composition, of the embodiments described herein, necessary or sufficient to achieve the desired effect. For example, in some embodiments, the desired effect may include, without limitation, medically therapeutic, cosmetically therapeutic and/or prophylactic treatment, as appropriate.

The term “inhibit,” “suppress,” “decrease,” “interfere,” and/or “reduce” (and like terms) generally refers to the act of reducing, either directly or indirectly, a function, activity, or behavior relative to the natural, expected, or average or relative to current conditions.

The term “improves” is used to convey that the present invention changes either the appearance, form, characteristics, structure, function and/or physical attributes of the skin to which it is being provided, applied or administered. “Improves” may also refer to the overall physical state of an individual to whom an active agent has been administered. For example, an individual may feel that their skin has “improved” by administration of a composition containing an active agent.

In each of the embodiments disclosed herein, the compositions and methods may be utilized with or on a subject in need of such treatment, which may also be referred to as “in need thereof” As used herein, the phrase “in need thereof” means that the subject has been identified as having a need for the particular method or treatment and that the treatment has been given to the subject for that particular purpose.

As used herein, the term “pharmaceutically acceptable” and grammatical variations thereof, as they refer to carriers, diluents, excipients, reagents or other ingredients of the composition, represent that the materials used in the final composition are not irritating or otherwise harmful to the patient in general and to the skin, in particular, and preferably are pleasant and well tolerated with respect to general appearance, pH, color, smell and texture (feel), that they are not, for example, unacceptably sticky (tacky), oily or drying, and that they do spread easily, absorb into the skin at an acceptable rate of absorption. Such carriers, diluents, excipients, reagents or other ingredients are all suitable for topical administration.

The term “sebaceous gland” includes unilobular or multilobular glands that secrete sebum. Sebaceous glands include pilosebaceous units, fordyce spots, Meibomian glands, glands of the Zeiss and Montgomery areolar tubercles.

The phrase “disorder associated with sebaceous glands” includes diseases, conditions and symptoms related to sebaceous gland. Disorders associated with sebaceous glands include acne, seborrhea, sebaceoma, sebaceous carcinoma, seborrheic dermatitis, sebaceous cysts, sebaceous adenoma and sebaceous hyperplasia. Diseases or disorders associated with sebaceous glands, including inflammatory and non-inflammatory acne, follicular hyperkeratinization, seborrheic cysts, seborrhea, sebostasis, seborrheic dermatitis, sebaceous adenomas, sebaceous hyperplasia, and excess sebum production may be treated by compositions described herein. Additional skin conditions treated with the topical compositions described herein are selected from the group consisting of dermatitis, atopic dermatitis, seborrheic dermatitis, alopecia, contact dermatitis, psoriasis, urticaria eczema, burns, sunburn, pancreatitis, hepatitis, lichen planus, scleritis, scleroderma, dermatomyositis, itching associated with any of the preceding conditions, and any combination thereof.

The term “seborrhea” includes oily skin.

The term “seborrheic dermatitis” includes inflammatory skin disorders characterized by scaly, flaky, itchy, and red skin and includes seborrheic dermatitis caused by fungal, genetic, environmental, hormonal and immune function disorders.

The term “sebaceous cysts” include steatocystoma simplex (e.g., simple sebaceous duct cyst and solitary steatocystoma) and steatocystoma multiplex (e.g., epidermal polycystic disease and sebocystomatosis).

The term “sebaceous hyperplasia” includes enlargement of the sebaceous glands.

The term “skin” as used herein refers to the organ of the body which protects the subject from environmental irritations, regulates the body's temperature and allows for external sensations. The “skin” is separated into three layers: the outermost layer called the epidermis which contains melanocytes; the dermis which contains connective tissue, hair follicles and sweat glands; and the deepest subcutaneous layer called the hypodermis which is made up of fat and connective tissue.

As used herein, the term “topically” and “topical” refers to application of the compositions of the present invention to the surface of the skin and mucous membranes.

“Topical application” or “topical administration” refers to the delivery of a composition, for treating conditions of the epidermis or dermis, wherein the topical composition is applied to the skin and acts locally and does not have a systemic effect. Topical administration of a drug may often be advantageously applied in, for example, the treatment of various skin disorders.

As used herein the terms “topical formulations” and “topical compositions” refer to formulations or compositions that may be applied to skin or mucous membranes. Topical formulations or compositions may, for example, be used to confer therapeutic benefit to a patient or cosmetic benefit to a consumer. Such topical formulations or compositions may be provided in the form of a cream, foam, gel, lotion, or ointment.

The terms “treat,” “treated,” or “treating” as used herein refers to therapeutic treatment, cosmetic treatment and/or prophylactic or preventative measures, wherein the object is to prevent, reduce, eliminate or slow down (lessen) an undesired physiological condition, disorder or disease, or to obtain beneficial or desired clinical results (e.g. decrease acne, comedones, pimples, or breakouts). For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of unwanted side effects.

When used herein, the term “Tween 80” is the trademark name for polysorbate 80.

When used herein, the terms “Transcutol,” “Transcutol P,” and “Transcutol HP” are the trademark names for 2-(2-ethoxyethoxy)ethanol, also known as diethylene glycol monoethyl ether

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

All numerical ranges describing the weight percent of any component, excluding the API, includes an additional 5.0% of the disclosed range, an additional 4.0% of the disclosed range, an additional 3.0% of the disclosed range, an additional 2.0% of the disclosed range, an additional 1.0% of the disclosed range, and an additional 0.5% of the disclosed range.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability.

Embodiments herein are directed to topical compositions comprising 2-(4-(4-(6-carbamoyl-3,5-dimethylpyrazin-2-yl)phenyl)cyclohexyl)acetic acid, benzyl alcohol, diisopropyl adipate, diethyl sebacate, dimethyl isosorbide, 2-(2-ethoxyethoxy)ethanol, hexylene glycol, polysorbate 80, and optionally one or more pharmaceutically acceptable topical excipients such as but not limited to PEG 400, ethanol, isopropanol. In some embodiments, 2-(4-(4-(6-carbamoyl-3,5-dimethylpyrazin-2-yl)phenyl)cyclohexyl)acetic acid is present in the topical composition in a therapeutically effective amount. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, benzyl alcohol is at a concentration from about 0.5% to about 5.0% by weight of the final composition. In some embodiments, PEG 400 is at a concentration from about 1.0% to about 10.0% by weight of the final composition. In some embodiments, diisopropyl adipate is at a concentration from about 1.0% to about 20.0% by weight of the final composition. In some embodiments, diethyl sebacate is at a concentration from about 1.0% to about 20.0% by weight of the final composition. In some embodiments, dimethyl isosorbide is at a concentration from about 1.0% to about 20.0% by weight of the final composition. In some embodiments, 2-(2-ethoxyethoxy)ethanol is at a concentration from about 1.0% to about 30.0% by weight of the final composition. In some embodiments, hexylene glycol is at a concentration from about 1.0% to about 15.0% by weight of the final composition. In some embodiments, polysorbate 80 is at a concentration from about 1.0% to about 10.0% by weight of the final composition.

In certain embodiments, the topical composition comprises 2-(4-(4-(6-carbamoyl-3,5-dimethylpyrazin-2-yl)phenyl)cyclohexyl)acetic acid at about 0.6% by weight, benzyl alcohol at about 2.7% by weight, PEG 400 at about 7.3% by weight, diisopropyl adipate at about 15.0% by weight, diethyl sebacate at about 15.0% by weight, dimethyl isosorbide at about 15.0% by weight, 2-(2-ethoxyethoxy)ethanol at about 25.0% by weight, hexylene glycol at about 12.0% by weight, polysorbate 80 at about 8.0% by weight, and optionally one or more pharmaceutically acceptable topical excipients.

Embodiments herein are directed to topical compositions comprising 2-(4-(4-(6-carbamoyl-3,5-dimethylpyrazin-2-yl)phenyl)cyclohexyl)acetic acid, benzyl alcohol, PEG 400, 2-(2-ethoxyethoxy)ethanol, polysorbate 80, and optionally one or more pharmaceutically acceptable topical excipients. In some embodiments, the topical composition further comprises diisopropyl adipate. In some embodiments, the topical composition further comprises diethyl sebacate. In some embodiments, the topical composition further comprises dimethyl isosorbide. In some embodiments, the topical composition further comprises hexylene glycol. In some embodiments, 2-(4-(4-(6-carbamoyl-3,5-dimethylpyrazin-2-yl)phenyl)cyclohexyl)acetic acid is present in the topical composition in a therapeutically effective amount. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, benzyl alcohol is at a concentration from about 0.5% to about 5.0% by weight of the final composition. In some embodiments, PEG 400 is at a concentration from about 30.0% to about 60.0% by weight of the final composition. In some embodiments, 2-(2-ethoxyethoxy)ethanol is at a concentration from about 1.0% to about 30.0% by weight of the final composition. In some embodiments, polysorbate 80 is at a concentration from about 1.0% to about 10.0% by weight of the final composition. In some embodiments, the composition further comprises diisopropyl adipate in a concentration from about 1.0% to about 25.0% by weight of the final composition. In some embodiments, the composition further comprises diethyl sebacate in a concentration from about 1.0% to about 30.0% by weight of the final composition. In some embodiments, the composition further comprises dimethyl isosorbide in a concentration from about 1.0% to about 20.0% by weight of the final composition. In some embodiments, the composition further comprises hexylene glycol in a concentration from about 1.0% to about 20.0% by weight of the final composition.

In certain embodiments, the topical composition comprises 2-(4-(4-(6-carbamoyl-3,5-dimethylpyrazin-2-yl)phenyl)cyclohexyl)acetic acid at about 0.6% by weight, benzyl alcohol at about 2.7% by weight, PEG 400 at about 44.3% by weight, 2-(2-ethoxyethoxy)ethanol at about 25.0% by weight, polysorbate 80 at about 8.0% by weight, diisopropyl adipate at about 20.0% by weight, and optionally one or more pharmaceutically acceptable topical excipients.

In certain embodiments, the topical composition comprises 2-(4-(4-(6-carbamoyl-3,5-dimethylpyrazin-2-yl)phenyl)cyclohexyl)acetic acid at about 0.6% by weight, benzyl alcohol at about 2.7% by weight, PEG 400 at about 40.3% by weight, 2-(2-ethoxyethoxy)ethanol at about 25.0% by weight, polysorbate 80 at about 8.0% by weight, diethyl sebacate at about 24.0% by weight, and optionally one or more pharmaceutically acceptable topical excipients.

In certain embodiments, the topical composition comprises 2-(4-(4-(6-carbamoyl-3,5-dimethylpyrazin-2-yl)phenyl)cyclohexyl)acetic acid at about 0.6% by weight, benzyl alcohol at about 2.7% by weight, PEG 400 at about 49.3% by weight, 2-(2-ethoxyethoxy)ethanol at about 25.0% by weight, polysorbate 80 at about 8% by weight, dimethyl isosorbide at about 15.0% by weight, and optionally one or more pharmaceutically acceptable topical excipients.

In certain embodiments, the topical composition comprises 2-(4-(4-(6-carbamoyl-3,5-dimethylpyrazin-2-yl)phenyl)cyclohexyl)acetic acid at about 0.6% by weight, benzyl alcohol at about 2.7% by weight, PEG 400 at about 52.3% by weight, 2-(2-ethoxyethoxy)ethanol at about 25.0% by weight, polysorbate 80 at about 8.0% by weight, hexylene glycol at about 12.0% by weight, and optionally one or more pharmaceutically acceptable topical excipients.

Embodiments herein are directed to topical compositions comprising 2-(4-(4-(6-carbamoyl-3,5-dimethylpyrazin-2-yl)phenyl)cyclohexyl)acetic acid, PEG 400, dimethyl isosorbide, 2-(2-ethoxyethoxy)ethanol, DMSO, and optionally one or more pharmaceutically acceptable topical excipients. In some embodiments, 2-(4-(4-(6-carbamoyl-3,5-dimethylpyrazin-2-yl)phenyl)cyclohexyl)acetic acid is present in the topical composition in a therapeutically effective amount. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, the PEG 400 is at a concentration from about 20.0% to about 30.0% by weight of the final composition. In some embodiments, the dimethyl isosorbide at a concentration from about 1.0% to about 30.0% by weight of the final composition. In some embodiments, the 2-(2-ethoxyethoxy)ethanol is at a concentration from about 1.0% to about 30.0% by weight of the final composition. In some embodiments, the DMSO is at a concentration from about 10.0% to about 30.0% by weight of the final composition.

In certain embodiments, the topical composition comprises 2-(4-(4-(6-carbamoyl-3,5-dimethylpyrazin-2-yl)phenyl)cyclohexyl)acetic acid at about 0.6% by weight, PEG 400 at about 25.0% by weight, dimethyl isosorbide at about 25.0% by weight, 2-(2-ethoxyethoxy)ethanol at about 25.0% by weight, DMSO at about 25.0% by weight, and optionally one or more pharmaceutically acceptable topical excipients.

Embodiments herein are directed to topical compositions comprising 2-(4-(4-(6-carbamoyl-3,5-dimethylpyrazin-2-yl)phenyl)cyclohexyl)acetic acid, propylene glycol, oleic acid, polysorbate 80, isopropyl myristate, phenoxyethanol, and optionally one or more pharmaceutically acceptable topical excipients. In some embodiments, 2-(4-(4-(6-carbamoyl-3,5-dimethylpyrazin-2-yl)phenyl)cyclohexyl)acetic acid is present in the topical composition in a therapeutically effective amount. In some embodiments, the 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, propylene glycol is at a concentration from about 30.0% to about 50.0% by weight of the final composition. In some embodiments, the oleic acid is at a concentration from about 15.0% to about 30.0% by weight of the final composition. In some embodiments, polysorbate 80 is at a concentration from about 1.0% to about 10.0% by weight of the final composition. In some embodiments, isopropyl myristate is at a concentration from about 15.0% to about 30.0% by weight of the final composition. In some embodiments, phenoxyethanol is at a concentration from about 0.1% to about 5.0% by weight of the final composition.

In certain embodiments, the topical composition comprises 2-(4-(4-(6-carbamoyl-3,5-dimethylpyrazin-2-yl)phenyl)cyclohexyl)acetic acid at about 0.6% by weight, propylene glycol at about 42.0% by weight, oleic acid at about 25.0% by weight, polysorbate 80 at about 8.0% by weight, isopropyl myristate at about 25.0% by weight, phenoxyethanol at about 1.0% by weight, and optionally one or more pharmaceutically acceptable topical excipients.

Embodiments herein are directed to topical compositions comprising 2-(4-(4-(6-carbamoyl-3,5-dimethylpyrazin-2-yl)phenyl)cyclohexyl)acetic acid, benzyl alcohol, glycerin, and optionally one or more pharmaceutically acceptable topical excipients. In some embodiments, 2-(4-(4-(6-carbamoyl-3,5-dimethylpyrazin-2-yl)phenyl)cyclohexyl)acetic acid is present in the topical composition in a therapeutically effective amount. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, benzyl alcohol is at a concentration from about 25.0% to about 60.0% by weight of the final composition. In some embodiments, glycerin is at a concentration from about 25.0% to about 60.0% by weight of the final composition.

In certain embodiments, the topical composition comprises 2-(4-(4-(6-carbamoyl-3,5-dimethylpyrazin-2-yl)phenyl)cyclohexyl)acetic acid at about 0.6% by weight, benzyl alcohol at about 50.0% by weight, glycerin at about 50.0% by weight, and optionally one or more pharmaceutically acceptable topical excipients.

Embodiments herein are directed to topical compositions comprising 2-(4-(4-(6-carbamoyl-3,5-dimethylpyrazin-2-yl)phenyl)cyclohexyl)acetic acid, DMSO, water, and optionally one or more pharmaceutically acceptable topical excipients. In some embodiments, 2-(4-(4-(6-carbamoyl-3,5-dimethylpyrazin-2-yl)phenyl)cyclohexyl)acetic acid is present in the topical composition in a therapeutically effective amount. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, DMSO is at a concentration from about 50.0% to about 75.0% by weight of the final composition. In some embodiments, water is at a concentration from about 20.0% to about 35.0% by weight of the final composition.

In certain embodiments, the topical composition comprises 2-(4-(4-(6-carbamoyl-3,5-dimethylpyrazin-2-yl)phenyl)cyclohexyl)acetic acid at about 0.6% by weight, DMSO at about 70.0% by weight, water at about 30.0% by weight, and optionally one or more pharmaceutically acceptable topical excipients.

Embodiments herein are directed to topical compositions comprising 2-(4-(4-(6-carbamoyl-3,5-dimethylpyrazin-2-yl)phenyl)cyclohexyl)acetic acid, propylene glycol, water, and optionally one or more pharmaceutically acceptable topical excipients. In some embodiments, 2-(4-(4-(6-carbamoyl-3,5-dimethylpyrazin-2-yl)phenyl)cyclohexyl)acetic acid is present in the topical composition in a therapeutically effective amount. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, propylene glycol is at a concentration from about 50.0% to about 75.0% by weight of the final composition. In some embodiments, water is at a concentration from about 20.0% to about 35.0% by weight of the final composition.

In certain embodiments, the topical composition comprises 2-(4-(4-(6-carbamoyl-3,5-dimethylpyrazin-2-yl)phenyl)cyclohexyl)acetic acid at about 0.6% by weight, propylene glycol at about 70.0% by weight, water at about 30.0% by weight, and optionally one or more pharmaceutically acceptable topical excipients.

Embodiments herein are directed to topical compositions comprising 2-(4-(4-(6-carbamoyl-3,5-dimethylpyrazin-2-yl)phenyl)cyclohexyl)acetic acid, propylene glycol, glycerin, and optionally one or more pharmaceutically acceptable topical excipients. In some embodiments, 2-(4-(4-(6-carbamoyl-3,5-dimethylpyrazin-2-yl)phenyl)cyclohexyl)acetic acid is present in the topical composition in a therapeutically effective amount. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, propylene glycol is at a concentration from about 50.0% to about 75.0% by weight of the final composition. In some embodiments, glycerin is at a concentration from about 20.0% to about 35.0% by weight of the final composition.

In certain embodiments, the topical composition comprises 2-(4-(4-(6-carbamoyl-3,5-dimethylpyrazin-2-yl)phenyl)cyclohexyl)acetic acid at about 0.6% by weight, propylene glycol at about 70.0% by weight, glycerin at about 30.0% by weight, and optionally one or more pharmaceutically acceptable topical excipients.

Embodiments herein are directed to topical compositions comprising 2-(4-(4-(6-carbamoyl-3,5-dimethylpyrazin-2-yl)phenyl)cyclohexyl)acetic acid, propylene glycol, 2-(2-ethoxyethoxy)ethanol, PEG400, butylated hydroxytoluene, and optionally one or more pharmaceutically acceptable topical excipients. In some embodiments, the topical composition further comprises glycerin. In some embodiments, the topical composition further comprises cellulose polymers. In some embodiments, the topical composition further comprises Carbomer Homopolymer Type A, B, or C. In some embodiments, the topical composition further comprises Carbomer Homopolymer Type B. In some embodiments, the topical composition further comprises Poloxamer. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, propylene glycol is at a concentration from about 10.0% to about 50.0% by weight of the final composition. In some embodiments, 2-(2-ethoxyethoxy)ethanol is at a concentration from about 30.0% to about 75.0% by weight of the final composition. In some embodiments, PEG400 is at a concentration from about 15.0% to about 50.0% by weight of the final composition. In some embodiments, glycerin is at a concentration from about 5.0% to about 25.0% by weight of the final composition. In some embodiments, butylated hydroxytoluene is at a concentration from 0.01% to about 5.0% by weight of the final composition. In some embodiments, cellulose polymers is at a concentration from about 0.1% to about 5.0% by weight of the final composition. In some embodiments, Carbomer Homopolymer Type B is at a concentration from about 0.1% to about 5.0% by weight of the final composition.

In certain embodiments, the topical composition comprises 2-(4-(4-(6-carbamoyl-3,5-dimethylpyrazin-2-yl)phenyl)cyclohexyl)acetic acid at about 0.5% by weight, propylene glycol at about 20.0% by weight, 2-(2-ethoxyethoxy)ethanol at about 42.9% by weight, PEG400 at about 25.0% by weight, glycerin at about 10.0% by weight, cellulose polymers at about 1.5% by weight, butylated hydroxytoluene at about 0.1% by weight, and optionally one or more pharmaceutically acceptable topical excipients.

In certain embodiments, the topical composition comprises 2-(4-(4-(6-carbamoyl-3,5-dimethylpyrazin-2-yl)phenyl)cyclohexyl)acetic acid at about 0.6% by weight, propylene glycol at about 20.0% by weight, 2-(2-ethoxyethoxy)ethanol at about 42.8% by weight, PEG400 at about 35.0% by weight, Carbomer Homopolymer Type B at about 1.0% by weight, butylated hydroxytoluene at about 0.1% by weight, and optionally one or more pharmaceutically acceptable topical excipients.

In certain embodiments, the topical composition comprises 2-(4-(4-(6-carbamoyl-3,5-dimethylpyrazin-2-yl)phenyl)cyclohexyl)acetic acid at about 0.6% by weight, propylene glycol at about 20.0% by weight, 2-(2-ethoxyethoxy)ethanol at about 42.8% by weight, PEG400 at about 35.0% by weight, cellulose polymers at about 1.5% by weight, butylated hydroxytoluene at about 0.1% by weight, and optionally one or more pharmaceutically acceptable topical excipients.

Embodiments herein are directed to topical compositions comprising 2-(4-(4-(6-carbamoyl-3,5-dimethylpyrazin-2-yl)phenyl)cyclohexyl)acetic acid, 2-(2-ethoxyethoxy)ethanol, PEG400, glycerin, cellulose polymers, butylated hydroxytoluene, and optionally one or more pharmaceutically acceptable topical excipients. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, 2-(2-ethoxyethoxy)ethanol is at a concentration from about 30.0% to about 75.0% by weight of the final composition. In some embodiments, PEG400 is at a concentration from about 15.0% to about 65.0% by weight of the final composition. In some embodiments, glycerin is at a concentration from about 5.0% to about 25.0% by weight of the final composition. In some embodiments, butylated hydroxytoluene is at a concentration from 0.01% to about 5.0% by weight of the final composition. In some embodiments, cellulose polymers is at a concentration from about 0.1% to about 5.0% by weight of the final composition.

In certain embodiments, the topical composition comprises 2-(4-(4-(6-carbamoyl-3,5-dimethylpyrazin-2-yl)phenyl)cyclohexyl)acetic acid at about 0.6% by weight, 2-(2-ethoxyethoxy)ethanol at about 42.8% by weight, PEG400 at about 45.0% by weight, glycerin at about 10.0% by weight, butylated hydroxytoluene at about 0.1% by weight, cellulose polymers at about 1.5% by weight, and optionally one or more pharmaceutically acceptable topical excipients.

Embodiments herein are directed to topical compositions comprising 2-(4-(4-(6-carbamoyl-3,5-dimethylpyrazin-2-yl)phenyl)cyclohexyl)acetic acid, propylene glycol, 2-(2-ethoxyethoxy)ethanol, glycerin, dimethyl isosorbide, butylated hydroxytoluene, and optionally one or more pharmaceutically acceptable topical excipients. In some embodiments, the topical composition further comprises cellulose polymers. In some embodiments, the topical composition further comprises Carbomer Homopolymer Type A, B, or C. In some embodiments, the topical composition further comprises Carbomer Homopolymer Type B. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, propylene glycol is at a concentration from about 20.0% to about 50.0% by weight of the final composition. In some embodiments, 2-(2-ethoxyethoxy)ethanol is at a concentration from about 25.0% to about 65.0% by weight of the final composition. In some embodiments, glycerin is at a concentration from about 5.0% to about 25.0% by weight of the final composition. In some embodiments, dimethyl isosorbide is at a concentration from about 5.0% to about 25.0% by weight of the final composition. In some embodiments, butylated hydroxytoluene is at a concentration from 0.01% to about 5.0% by weight of the final composition. In some embodiments, cellulose polymers is at a concentration from about 0.1% to about 5.0% by weight of the final composition. In some embodiments, Carbomer Homopolymer Type B is at a concentration from about 0.1% to about 5.0% by weight of the final composition.

In certain embodiments, the topical composition comprises comprising 2-(4-(4-(6-carbamoyl-3,5-dimethylpyrazin-2-yl)phenyl)cyclohexyl)acetic acid at about 0.6% by weight, propylene glycol at about 32% by weight, 2-(2-ethoxyethoxy)ethanol at 38.8% by weight, glycerin at about 12.0%, dimethyl isosorbide at about 15.0% by weight, cellulose polymers at about 1.5% by weight, butylated hydroxytoluene at about 0.1% by weight, and optionally one or more pharmaceutically acceptable topical excipients.

In certain embodiments, the topical composition comprises 2-(4-(4-(6-carbamoyl-3,5-dimethylpyrazin-2-yl)phenyl)cyclohexyl)acetic acid at about 0.6% by weight, propylene glycol at about 32.0% by weight, 2-(2-ethoxyethoxy)ethanol at 39.3% by weight, glycerin at about 12.0%, dimethyl isosorbide at about 15.0% by weight, Carbomer Homopolymer Type B at about 1.0% by weight, butylated hydroxytoluene at about 0.1% by weight, and optionally one or more pharmaceutically acceptable topical excipients.

Embodiments herein are directed to a topical composition comprising a therapeutically effective amount of 2-(4-(4-(6-carbamoyl-3,5-dimethylpyrazin-2-yl)phenyl)cyclohexyl)acetic acid and a pharmaceutically acceptable topical excipients, wherein 90% confidence interval for the ratio of means (population geometric means based on log-transformed data) of the AUC of the topical composition is within 80-125% of the AUC of any one the foregoing topical compositions and the 90% confidence internal for the ratio of means of the Cₘₐₓ of the topical composition is within 70-143% of the Cₘₐₓ of the same foregoing topical composition.

In all the foregoing embodiments, the one or more pharmaceutically acceptable topical excipients may be selected from the group consisting of propylene glycol, 2-(2-ethoxyethoxy)ethanol, PEG 400, Glycerin, Dimethyl isosorbide, HPC (such as HPC medium chain), Carbomer Homopolymer Types A, B, and C (such as Carbopol 974), dimethicone, and cyclomethicone.

Embodiments herein are directed to methods of treating skin conditions in a patient in need thereof comprising topically applying a topical composition comprising a therapeutically effective amount of 2-(4-(4-(6-carbamoyl-3,5-dimethylpyrazin-2-yl)phenyl)cyclohexyl)acetic acid, benzyl alcohol, diisopropyl adipate, diethyl sebacate, dimethyl isosorbide, 2-(2-ethoxyethoxy)ethanol, hexylene glycol, polysorbate 80, and optionally one or more pharmaceutically acceptable topical excipients such as but not limited to PEG 400, ethanol, isopropanol. In certain embodiments, the skin condition is selected from the group consisting of inflammatory acne, non-inflammatory acne, acne, comedonal acne, inflammatory acne, papular acne, pustular acne, cystic acne, cysts follicular hyperkeratinization, seborrheic cysts, seborrhea, sebostasis, seborrheic dermatitis, sebaceous adenomas, sebaceous hyperplasia, and excess sebum production, dermatitis, atopic dermatitis, seborrheic dermatitis, alopecia, contact dermatitis, psoriasis, urticaria eczema, burns, sunburn, pancreatitis, hepatitis, lichen planus, scleritis, scleroderma, dermatomyositis, itching associated with any of the preceding conditions, and any combination thereof.

In some embodiments, the topical composition comprises 2-(4-(4-(6-carbamoyl-3,5-dimethylpyrazin-2-yl)phenyl)cyclohexyl)acetic acid, benzyl alcohol, diisopropyl adipate, diethyl sebacate, dimethyl isosorbide, 2-(2-ethoxyethoxy)ethanol, hexylene glycol, polysorbate 80, and optionally one or more pharmaceutically acceptable topical excipients such as but not limited to PEG 400, ethanol, isopropanol. In some embodiments, 2-(4-(4-(6-carbamoyl-3,5-dimethylpyrazin-2-yl)phenyl)cyclohexyl)acetic acid is present in the topical composition in a therapeutically effective amount. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, benzyl alcohol is at a concentration from about 0.5% to about 5.0% by weight of the final composition. In some embodiments, PEG 400 is at a concentration from about 1.0% to about 10.0% by weight of the final composition. In some embodiments, diisopropyl adipate is at a concentration from about 1% to about 20.0% by weight of the final composition. In some embodiments, diethyl sebacate is at a concentration from about 1.0% to about 20.0% by weight of the final composition. In some embodiments, dimethyl isosorbide is at a concentration from about 1.0% to about 20.0% by weight of the final composition. In some embodiments, 2-(2-ethoxyethoxy)ethanol is at a concentration from about 1.0% to about 30.0% by weight of the final composition. In some embodiments, hexylene glycol is at a concentration from about 1.0% to about 15.0% by weight of the final composition. In some embodiments, polysorbate 80 is at a concentration from about 1.0% to about 10.0% by weight of the final composition. In certain embodiments, the topical composition comprises 2-(4-(4-(6-carbamoyl-3,5-dimethylpyrazin-2-yl)phenyl)cyclohexyl)acetic acid at about 0.6% by weight, benzyl alcohol at about 2.7% by weight, PEG 400 at about 7.3% by weight, diisopropyl adipate at about 15.0% by weight, diethyl sebacate at about 15.0%, dimethyl isosorbide at about 15.0% by weight, 2-(2-ethoxyethoxy)ethanol at about 25.0% by weight, hexylene glycol at about 12.0% by weight, polysorbate 80 at about 8.0% by weight, and optionally one or more pharmaceutically acceptable topical excipients.

The topical compositions of the present invention may be formulated by those skilled in the art as liquids, toners, solutions, sprays, emulsions, moisturizers, sunscreens, creams, lotions, masks, suspensions, triturates, gels, jellies, pastes, foams, ointments, shampoos, adhesives, serums, treated clothes or pads and the like. In some embodiments the topical composition is formulated as eye drops, as ear drops, or as a composition which can be applied to the surface of the tooth.

In embodiments described herein, the topical compositions may be applied to the skin by any means known in the art including, but not limited to, by an aerosol, spray, pump-pack, brush, swab, or other applicator. The applicator may provide either a fixed or variable metered dose application such as a metered dose aerosol, a stored-energy metered dose pump or a manual metered dose pump.

In embodiments described herein, the topical composition is formulated as to be applied to a site one time a day or multiple times per day.

Embodiments herein are directed to methods of treating skin conditions in a patient in need thereof comprising topically applying a topical composition comprising 2-(4-(4-(6-carbamoyl-3,5-dimethylpyrazin-2-yl)phenyl)cyclohexyl)acetic acid, benzyl alcohol, PEG 400, 2-(2-ethoxyethoxy)ethanol, polysorbate 80, and optionally one or more pharmaceutically acceptable topical excipients. In certain embodiments, the skin condition is selected from the group consisting of inflammatory acne, non-inflammatory acne, acne, comedonal acne, inflammatory acne, papular acne, pustular acne, cystic acne, follicular hyperkeratinization, seborrheic cysts, seborrhea, sebostasis, seborrheic dermatitis, sebaceous adenomas, sebaceous hyperplasia, and excess sebum production, dermatitis, atopic dermatitis, seborrheic dermatitis, alopecia, contact dermatitis, psoriasis, urticaria eczema, burns, sunburn, pancreatitis, hepatitis, lichen planus, scleritis, scleroderma, dermatomyositis, itching associated with any of the preceding conditions, and any combination thereof. Embodiments herein are directed to topical compositions comprising 2-(4-(4-(6-carbamoyl-3,5-dimethylpyrazin-2-yl)phenyl)cyclohexyl)acetic acid, benzyl alcohol, PEG 400, 2-(2-ethoxyethoxy)ethanol, polysorbate 80, and optionally one or more pharmaceutically acceptable topical excipients. In some embodiments, the topical composition further comprises diisopropyl adipate. In some embodiments, the topical composition further comprises diethyl sebacate. In some embodiments, the topical composition further comprises dimethyl isosorbide. In some embodiments, the topical composition further comprises hexylene glycol. In some embodiments, 2-(4-(4-(6-carbamoyl-3,5-dimethylpyrazin-2-yl)phenyl)cyclohexyl)acetic acid is present in the topical composition in a therapeutically effective amount. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, benzyl alcohol is at a concentration from about 0.5% to about 5.0% by weight of the final composition. In some embodiments, PEG 400 is at a concentration from about 30.0% to about 60.0% by weight of the final composition. In some embodiments, 2-(2-ethoxyethoxy)ethanol is at a concentration from about 1.0% to about 30.0% by weight of the final composition. In some embodiments, polysorbate 80 is at a concentration from about 1.0% to about 10.0% by weight of the final composition. In some embodiments, the composition further comprises diisopropyl adipate in a concentration from about 1.0% to about 25.0% by weight of the final composition. In some embodiments, the composition further comprises diethyl sebacate in a concentration from about 1.0% to about 30.0% by weight of the final composition. In some embodiments, the composition further comprises dimethyl isosorbide in a concentration from about 1.0% to about 20.0% by weight of the final composition. In some embodiments, the composition further comprises hexylene glycol in a concentration from about 1.0% to about 20.0% by weight of the final composition. In certain embodiments, the topical composition comprises 2-(4-(4-(6-carbamoyl-3,5-dimethylpyrazin-2-yl)phenyl)cyclohexyl)acetic acid at about 2.0% by weight, benzyl alcohol at about 2.7% by weight, PEG 400 at about 44.3% by weight, 2-(2-ethoxyethoxy)ethanol at about 25.0% by weight, polysorbate 80 at about 8.0% by weight, diisopropyl adipate at about 20.0% by weight, and optionally one or more pharmaceutically acceptable topical excipients. In certain embodiments, the topical composition comprises 2-(4-(4-(6-carbamoyl-3,5-dimethylpyrazin-2-yl)phenyl)cyclohexyl)acetic acid at about 2% by weight, benzyl alcohol at about 2.7% by weight, PEG 400 at about 40.3% by weight, 2-(2-ethoxyethoxy)ethanol at about 25.0% by weight, polysorbate 80 at about 8.0% by weight, diethyl sebacate at about 24.0% by weight, and optionally one or more pharmaceutically acceptable topical excipients. In certain embodiments, the topical composition comprises 2-(4-(4-(6-carbamoyl-3,5-dimethylpyrazin-2-yl)phenyl)cyclohexyl)acetic acid at about 2.0% by weight, benzyl alcohol at about 2.7% by weight, PEG 400 at about 49.3% by weight, 2-(2-ethoxyethoxy)ethanol at about 25.0% by weight, polysorbate 80 at about 8.0% by weight, dimethyl isosorbide at about 15.0% by weight, and optionally one or more pharmaceutically acceptable topical excipients. In certain embodiments, the topical composition comprises 2-(4-(4-(6-carbamoyl-3,5-dimethylpyrazin-2-yl)phenyl)cyclohexyl)acetic acid at about 2.0% by weight, benzyl alcohol at about 2.7% by weight, PEG 400 at about 52.3% by weight, 2-(2-ethoxyethoxy)ethanol at about 25.0% by weight, polysorbate 80 at about 8.0% by weight, hexylene glycol at about 12.0% by weight, and optionally one or more pharmaceutically acceptable topical excipients.

Embodiments herein are directed to methods of treating skin conditions in a patient in need thereof comprising topically applying a topical composition comprising 2-(4-(4-(6-carbamoyl-3,5-dimethylpyrazin-2-yl)phenyl)cyclohexyl)acetic acid, PEG 400, dimethyl isosorbide, 2-(2-ethoxyethoxy)ethanol, DMSO, and optionally one or more pharmaceutically acceptable topical excipients. In certain embodiments, the skin condition is selected from the group consisting of inflammatory acne, non-inflammatory acne, acne, comedonal acne, inflammatory acne, papular acne, pustular acne, cystic acne, follicular hyperkeratinization, seborrheic cysts, seborrhea, sebostasis, seborrheic dermatitis, sebaceous adenomas, sebaceous hyperplasia, and excess sebum production, dermatitis, atopic dermatitis, seborrheic dermatitis, alopecia, contact dermatitis, psoriasis, urticaria eczema, burns, sunburn, pancreatitis, hepatitis, lichen planus, scleritis, scleroderma, dermatomyositis, itching associated with any of the preceding conditions, and any combination thereof. Embodiments herein are directed to topical compositions comprising 2-(4-(4-(6-carbamoyl-3,5-dimethylpyrazin-2-yl)phenyl)cyclohexyl)acetic acid, PEG 400, dimethyl isosorbide, 2-(2-ethoxyethoxy)ethanol, DMSO, and optionally one or more pharmaceutically acceptable topical excipients. In some embodiments, 2-(4-(4-(6-carbamoyl-3,5-dimethylpyrazin-2-yl)phenyl)cyclohexyl)acetic acid is present in the topical composition in a therapeutically effective amount. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, the PEG 400 is at a concentration from about 20.0% to about 30.0% by weight of the final composition. In some embodiments, the dimethyl isosorbide at a concentration from about 1.0% to about 30.0% by weight of the final composition. In some embodiments, the 2-(2-ethoxyethoxy)ethanol is at a concentration from about 1.0% to about 30.0% by weight of the final composition. In some embodiments, the DMSO is at a concentration from about 10.0% to about 30.0% by weight of the final composition. In certain embodiments, the topical composition comprises 2-(4-(4-(6-carbamoyl-3,5-dimethylpyrazin-2-yl)phenyl)cyclohexyl)acetic acid at about 2.0% by weight, PEG 400 at about 25.0% by weight, dimethyl isosorbide at about 25.0% by weight, 2-(2-ethoxyethoxy)ethanol at about 25.0% by weight, DMSO at about 25.0% by weight, and optionally one or more pharmaceutically acceptable topical excipients.

Embodiments herein are directed to methods of treating skin conditions in a patient in need thereof comprising topically applying a topical composition comprising 2-(4-(4-(6-carbamoyl-3,5-dimethylpyrazin-2-yl)phenyl)cyclohexyl)acetic acid, propylene glycol, oleic acid, polysorbate 80, isopropyl myristate, phenoxyethanol, and optionally one or more pharmaceutically acceptable topical excipients. In certain embodiments, the skin condition is selected from the group consisting of inflammatory acne, non-inflammatory acne, acne, comedonal acne, inflammatory acne, papular acne, pustular acne, cystic acne, follicular hyperkeratinization, seborrheic cysts, seborrhea, sebostasis, seborrheic dermatitis, sebaceous adenomas, sebaceous hyperplasia, and excess sebum production, dermatitis, atopic dermatitis, seborrheic dermatitis, alopecia, contact dermatitis, psoriasis, urticaria eczema, burns, sunburn, pancreatitis, hepatitis, lichen planus, scleritis, scleroderma, dermatomyositis, itching associated with any of the preceding conditions, and any combination thereof. Embodiments herein are directed to topical compositions comprising 2-(4-(4-(6-carbamoyl-3,5-dimethylpyrazin-2-yl)phenyl)cyclohexyl)acetic acid, propylene glycol, oleic acid, polysorbate 80, isopropyl myristate, phenoxyethanol, and optionally one or more pharmaceutically acceptable topical excipients. In some embodiments, 2-(4-(4-(6-carbamoyl-3,5-dimethylpyrazin-2-yl)phenyl)cyclohexyl)acetic acid is present in the topical composition in a therapeutically effective amount. In some embodiments, the 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, propylene glycol is at a concentration from about 30.0% to about 50.0% by weight of the final composition. In some embodiments, the oleic acid is at a concentration from about 15.0% to about 30.0% by weight of the final composition. In some embodiments, polysorbate 80 is at a concentration from about 1.0% to about 10.0% by weight of the final composition. In some embodiments, isopropyl myristate is at a concentration from about 15.0% to about 30.0% by weight of the final composition. In some embodiments, phenoxyethanol is at a concentration from about 0.1% to about 5.0% by weight of the final composition. In certain embodiments, the topical composition comprises 2-(4-(4-(6-carbamoyl-3,5-dimethylpyrazin-2-yl)phenyl)cyclohexyl)acetic acid at about 0.6% by weight, propylene glycol at about 42.0% by weight, oleic acid at about 25.0% by weight, polysorbate 80 at about 8.0% by weight, isopropyl myristate at about 25.0% by weight, phenoxyethanol at about 1.0% by weight, and optionally one or more pharmaceutically acceptable topical excipients.

Embodiments herein are directed to methods of treating skin conditions in a patient in need thereof comprising topically applying a topical composition comprising 2-(4-(4-(6-carbamoyl-3,5-dimethylpyrazin-2-yl)phenyl)cyclohexyl)acetic acid, benzyl alcohol, glycerin, and optionally one or more pharmaceutically acceptable topical excipients. In certain embodiments, the skin condition is selected from the group consisting of inflammatory acne, non-inflammatory acne, acne, comedonal acne, inflammatory acne, papular acne, pustular acne, cystic acne, follicular hyperkeratinization, seborrheic cysts, seborrhea, sebostasis, seborrheic dermatitis, sebaceous adenomas, sebaceous hyperplasia, and excess sebum production, dermatitis, atopic dermatitis, seborrheic dermatitis, alopecia, contact dermatitis, psoriasis, urticaria eczema, burns, sunburn, pancreatitis, hepatitis, lichen planus, scleritis, scleroderma, dermatomyositis, itching associated with any of the preceding conditions, and any combination thereof. Embodiments herein are directed to a topical composition comprising 2-(4-(4-(6-carbamoyl-3,5-dimethylpyrazin-2-yl)phenyl)cyclohexyl)acetic acid, benzyl alcohol, glycerin, and optionally one or more pharmaceutically acceptable topical excipients. In some embodiments, 2-(4-(4-(6-carbamoyl-3,5-dimethylpyrazin-2-yl)phenyl)cyclohexyl)acetic acid is present in the topical composition in a therapeutically effective amount. In some embodiments, 2-(4-(4-(6-carbamoyl-3,5-dimethylpyrazin-2-yl)phenyl)cyclohexyl)acetic acid is at a concentration from about 0.1% to about 10.0% by weight of the final composition. In some embodiments, benzyl alcohol is at a concentration from about 25.0% to about 60.0% by weight of the final composition. In some embodiments, glycerin is at a concentration from about 25.0% to about 60.0% by weight of the final composition. In certain embodiments, the topical composition comprises 2-(4-(4-(6-carbamoyl-3,5-dimethylpyrazin-2-yl)phenyl)cyclohexyl)acetic acid at about 2.0% by weight, benzyl alcohol at about 50.0% by weight, glycerin at about 50.0% by weight, and optionally one or more pharmaceutically acceptable topical excipients.

Embodiments herein are directed to methods of treating skin conditions in a patient in need thereof comprising topically applying a topical composition comprising 2-(4-(4-(6-carbamoyl-3,5-dimethylpyrazin-2-yl)phenyl)cyclohexyl)acetic acid, DMSO, water, and optionally one or more pharmaceutically acceptable topical excipients. In certain embodiments, the skin condition is selected from the group consisting of inflammatory acne, non-inflammatory acne, acne, comedonal acne, inflammatory acne, papular acne, pustular acne, cystic acne, follicular hyperkeratinization, seborrheic cysts, seborrhea, sebostasis, seborrheic dermatitis, sebaceous adenomas, sebaceous hyperplasia, and excess sebum production, dermatitis, atopic dermatitis, seborrheic dermatitis, alopecia, contact dermatitis, psoriasis, urticaria eczema, burns, sunburn, pancreatitis, hepatitis, lichen planus, scleritis, scleroderma, dermatomyositis, itching associated with any of the preceding conditions, and any combination thereof. Embodiments herein are directed to a topical composition comprising 2-(4-(4-(6-carbamoyl-3,5-dimethylpyrazin-2-yl)phenyl)cyclohexyl)acetic acid, DMSO, water, and optionally one or more pharmaceutically acceptable topical excipients. In some embodiments, 2-(4-(4-(6-carbamoyl-3,5-dimethylpyrazin-2-yl)phenyl)cyclohexyl)acetic acid is present in the topical composition in a therapeutically effective amount. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, DMSO is at a concentration from about 50.0% to about 75.0% by weight of the final composition. In some embodiments, water is at a concentration from about 20.0% to about 35.0% by weight of the final composition. In certain embodiments, the topical composition comprises 2-(4-(4-(6-carbamoyl-3,5-dimethylpyrazin-2-yl)phenyl)cyclohexyl)acetic acid at about 0.6% by weight, DMSO at about 70.0% by weight, water at about 30.0% by weight, and optionally one or more pharmaceutically acceptable topical excipients.

Embodiments herein are directed to methods of treating skin conditions in a patient in need thereof comprising topically applying a topical composition comprising 2-(4-(4-(6-carbamoyl-3,5-dimethylpyrazin-2-yl)phenyl)cyclohexyl)acetic acid, propylene glycol, water, and optionally one or more pharmaceutically acceptable topical excipients. In certain embodiments, the skin condition is selected from the group consisting of inflammatory acne, non-inflammatory acne, acne, comedonal acne, inflammatory acne, papular acne, pustular acne, cystic acne, follicular hyperkeratinization, seborrheic cysts, seborrhea, sebostasis, seborrheic dermatitis, sebaceous adenomas, sebaceous hyperplasia, and excess sebum production, dermatitis, atopic dermatitis, seborrheic dermatitis, alopecia, contact dermatitis, psoriasis, urticaria eczema, burns, sunburn, pancreatitis, hepatitis, lichen planus, scleritis, scleroderma, dermatomyositis, itching associated with any of the preceding conditions, and any combination thereof. Embodiments herein are directed to a topical composition comprising 2-(4-(4-(6-carbamoyl-3,5-dimethylpyrazin-2-yl)phenyl)cyclohexyl)acetic acid, propylene glycol, water, and optionally one or more pharmaceutically acceptable topical excipients. In some embodiments, 2-(4-(4-(6-carbamoyl-3,5-dimethylpyrazin-2-yl)phenyl)cyclohexyl)acetic acid is present in the topical composition in a therapeutically effective amount. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, propylene glycol is at a concentration from about 50.0% to about 75.0% by weight of the final composition. In some embodiments, water is at a concentration from about 20.0% to about 35.0% by weight of the final composition. In certain embodiments, the topical composition comprises 2-(4-(4-(6-carbamoyl-3,5-dimethylpyrazin-2-yl)phenyl)cyclohexyl)acetic acid at about 0.6% by weight, propylene glycol at about 70.0% by weight, water at about 30.0% by weight, and optionally one or more pharmaceutically acceptable topical excipients.

Embodiments herein are directed to methods of treating skin conditions in a patient in need thereof comprising topically applying a topical composition comprising 2-(4-(4-(6-carbamoyl-3,5-dimethylpyrazin-2-yl)phenyl)cyclohexyl)acetic acid, propylene glycol, glycerin, and optionally one or more pharmaceutically acceptable topical excipients. In certain embodiments, the skin condition is selected from the group consisting of inflammatory acne, non-inflammatory acne, acne, comedonal acne, inflammatory acne, papular acne, pustular acne, cystic acne, follicular hyperkeratinization, seborrheic cysts, seborrhea, sebostasis, seborrheic dermatitis, sebaceous adenomas, sebaceous hyperplasia, and excess sebum production, dermatitis, atopic dermatitis, seborrheic dermatitis, alopecia, contact dermatitis, psoriasis, urticaria eczema, burns, sunburn, pancreatitis, hepatitis, lichen planus, scleritis, scleroderma, dermatomyositis, itching associated with any of the preceding conditions, and any combination thereof. Embodiments herein are directed to a topical composition comprising 2-(4-(4-(6-carbamoyl-3,5-dimethylpyrazin-2-yl)phenyl)cyclohexyl)acetic acid, propylene glycol, glycerin, and optionally one or more pharmaceutically acceptable topical excipients. In some embodiments, 2-(4-(4-(6-carbamoyl-3,5-dimethylpyrazin-2-yl)phenyl)cyclohexyl)acetic acid is present in the topical composition in a therapeutically effective amount. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, propylene glycol is at a concentration from about 50.0% to about 75.0% by weight of the final composition. In some embodiments, glycerin is at a concentration from about 20.0% to about 35.0% by weight of the final composition. In certain embodiments, the topical composition comprises 2-(4-(4-(6-carbamoyl-3,5-dimethylpyrazin-2-yl)phenyl)cyclohexyl)acetic acid at about 0.6% by weight, propylene glycol at about 70.0% by weight, glycerin at about 30.0% by weight, and optionally one or more pharmaceutically acceptable topical excipients.

Embodiments herein are directed to methods of treating skin conditions in a patient in need thereof comprising topically applying a topical composition comprising a therapeutically effective amount of 2-(4-(4-(6-carbamoyl-3,5-dimethylpyrazin-2-yl)phenyl)cyclohexyl)acetic acid, propylene glycol, 2-(2-ethoxyethoxy)ethanol, PEG400, butylated hydroxytoluene, and optionally one or more pharmaceutically acceptable topical excipients. In certain embodiments, the skin condition is selected from the group consisting of inflammatory acne, non-inflammatory acne, acne, comedonal acne, inflammatory acne, papular acne, pustular acne, cystic acne, follicular hyperkeratinization, seborrheic cysts, seborrhea, sebostasis, seborrheic dermatitis, sebaceous adenomas, sebaceous hyperplasia, and excess sebum production, dermatitis, atopic dermatitis, seborrheic dermatitis, alopecia, contact dermatitis, psoriasis, urticaria eczema, burns, sunburn, pancreatitis, hepatitis, lichen planus, scleritis, scleroderma, dermatomyositis, itching associated with any of the preceding conditions, and any combination thereof. In some embodiments, the topical composition comprises 2-(4-(4-(6-carbamoyl-3,5-dimethylpyrazin-2-yl)phenyl)cyclohexyl)acetic acid, propylene glycol, 2-(2-ethoxyethoxy)ethanol, PEG400, butylated hydroxytoluene, and optionally one or more pharmaceutically acceptable topical excipients. In some embodiments, the topical composition further comprises glycerin. In some embodiments, the topical composition further comprises cellulose polymers. In some embodiments, the topical composition further comprises Carbomer Homopolymer Type A, B or C (such as Carbopol 974). In some embodiments, the topical composition further comprises Poloxamer. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, propylene glycol is at a concentration from about 10.0% to about 50.0% by weight of the final composition. In some embodiments, 2-(2-ethoxyethoxy)ethanol is at a concentration from about 30.0% to about 75.0% by weight of the final composition. In some embodiments, PEG400 is at a concentration from about 15.0% to about 50.0% by weight of the final composition. In some embodiments, butylated hydroxytoluene is at a concentration from 0.01% to about 5.0% by weight of the final composition. In some embodiments, glycerin is at a concentration from about 5.0% to about 25.0% by weight of the final composition. In some embodiments, cellulose polymers is at a concentration from about 0.1% to about 5.0% by weight of the final composition. In some embodiments, Carbomer Homopolymer Type B is at a concentration from about 0.1% to about 5.0% by weight of the final composition.

Embodiments herein are directed to methods of treating skin conditions in a patient in need thereof comprising topically applying a topical composition comprising a therapeutically effective amount of 2-(4-(4-(6-carbamoyl-3,5-dimethylpyrazin-2-yl)phenyl)cyclohexyl)acetic acid, 2-(2-ethoxyethoxy)ethanol, PEG400, glycerin, cellulose polymers, butylated hydroxytoluene, and optionally one or more pharmaceutically acceptable topical excipients. In certain embodiments, the skin condition is selected from the group consisting of inflammatory acne, non-inflammatory acne, acne, comedonal acne, inflammatory acne, papular acne, pustular acne, cystic acne, follicular hyperkeratinization, seborrheic cysts, seborrhea, sebostasis, seborrheic dermatitis, sebaceous adenomas, sebaceous hyperplasia, and excess sebum production, dermatitis, atopic dermatitis, seborrheic dermatitis, alopecia, contact dermatitis, psoriasis, urticaria eczema, burns, sunburn, pancreatitis, hepatitis, lichen planus, scleritis, scleroderma, dermatomyositis, itching associated with any of the preceding conditions, and any combination thereof. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, 2-(2-ethoxyethoxy)ethanol is at a concentration from about 30.0% to about 75.0% by weight of the final composition. In some embodiments, PEG400 is at a concentration from about 15.0% to about 65.0% by weight of the final composition. In some embodiments, glycerin is at a concentration from about 5.0% to about 25.0% by weight of the final composition. In some embodiments, cellulose polymers is at a concentration from about 0.1% to about 5.0% by weight of the final composition. In some embodiments, butylated hydroxytoluene is at a concentration from 0.01% to about 5.0% by weight of the final composition.

Embodiments herein are directed to methods of treating skin conditions in a patient in need thereof comprising topically applying a topical composition comprising a therapeutically effective amount of 2-(4-(4-(6-carbamoyl-3,5-dimethylpyrazin-2-yl)phenyl)cyclohexyl)acetic acid, propylene glycol, 2-(2-ethoxyethoxy)ethanol, glycerin, dimethyl isosorbide, butylated hydroxytoluene, and optionally one or more pharmaceutically acceptable topical excipients. In certain embodiments, the skin condition is selected from the group consisting of inflammatory acne, non-inflammatory acne, acne, comedonal acne, inflammatory acne, papular acne, pustular acne, cystic acne, follicular hyperkeratinization, seborrheic cysts, seborrhea, sebostasis, seborrheic dermatitis, sebaceous adenomas, sebaceous hyperplasia, and excess sebum production, dermatitis, atopic dermatitis, seborrheic dermatitis, alopecia, contact dermatitis, psoriasis, urticaria eczema, burns, sunburn, pancreatitis, hepatitis, lichen planus, scleritis, scleroderma, dermatomyositis, itching associated with any of the preceding conditions, and any combination thereof. In some embodiments, the topical composition comprises 2-(4-(4-(6-carbamoyl-3,5-dimethylpyrazin-2-yl)phenyl)cyclohexyl)acetic acid, propylene glycol, 2-(2-ethoxyethoxy)ethanol, glycerin, dimethyl isosorbide, and optionally one or more pharmaceutically acceptable topical excipients. In some embodiments, the topical composition further comprises cellulose polymers. In some embodiments, the topical composition further comprises Carbomer Homopolymer Types A, B or C. In some embodiments, the topical composition further comprises Carbomer Homopolymer Type B. In some embodiments, 2-(4-(4-(6-carbamoyl-3,5-dimethylpyrazin-2-yl)phenyl)cyclohexyl)acetic acid is at a concentration from about 0.1% to about 1.0% by weight of the final composition. In some embodiments, propylene glycol is at a concentration from about 20.0% to about 50.0% by weight of the final composition. In some embodiments, 2-(2-ethoxyethoxy)ethanol is at a concentration from about 25.0% to about 65.0% by weight of the final composition. In some embodiments, glycerin is at a concentration from about 5.0% to about 25.0% by weight of the final composition. In some embodiments, dimethyl isosorbide is at a concentration from about 5.0% to about 25.0% by weight of the final composition. In some embodiments, butylated hydroxytoluene is at a concentration from 0.01% to about 5.0% by weight of the final composition. In some embodiments, cellulose polymers is at a concentration from about 0.1% to about 5.0% by weight of the final composition. In some embodiments, Carbomer Homopolymer Type B is at a concentration from about 0.1% to about 5.0% by weight of the final composition.

Embodiments herein are directed to methods of treating skin conditions in a patient in need thereof comprising topically applying a topical composition comprising a therapeutically effective amount of 2-(4-(4-(6-carbamoyl-3,5-dimethylpyrazin-2-yl)phenyl)cyclohexyl)acetic acid and a pharmaceutically acceptable topical excipients, wherein 90% confidence interval for the ratio of means (population geometric means based on log-transformed data) of the AUC of the topical composition is within 80-125% of the AUC of any one the foregoing topical compositions and the 90% confidence internal for the ratio of means of the Cₘₐₓ of the topical composition is within 70-143% of the Cₘₐₓ of the same foregoing topical composition.

In embodiments described herein, the method is directed to applying a topical composition once per day. In embodiments described herein, the method is directed to applying a topical composition multiple times per day. In some embodiments, the topical composition is applied two times per day, three times per day, four times per day, or five times per day. In some embodiments, the topical composition is applied one time in the morning and one time in the evening. In some embodiments, the topical composition is applied every 12 hours, every 11 hours, every 10 hours, every 9 hours, every 8 hours, every 7 hours, every 6 hours, every 5 hours, every 4 hours, every 3 hours, every 2 hours, or every hour.

In embodiments described herein, the method is directed to applying a topical composition to multiple sites on the skin of the body. For example, the topical composition may be applied over large areas of skin prophylactically or the topical composition may be applied to particular sites in need of treatment. In some embodiments, the topical composition is applied to the skin as a liquid, toner, solution, spray, emulsion, moisturizer, sunscreen, cream, lotion, mask, suspension, triturate, gel, jelly, paste, foam, ointment, shampoo, adhesive, serum, treated cloth or pad. In some embodiments, the topical composition is applied to the eyes as eye drops, placed in the ear canal as ear drops or to the surface of the tooth.

### EXAMPLES

### Example 1: Solubility and Stability of Formulations

Table 1 provides 11 topical formulations containing DMVT-503 and the relative solubility of each.

TABLE 1
Solubility of DMVT-503 in Prototype Formulation Solvent Systems
(ingredients provided as % w/w)

| | Mix | Mix | Mix | Mix | Mix | Mix | Mix | Mix | Mix | Mix | Mix |
|---|---|---|---|---|---|---|---|---|---|---|---|
| Ingredient | 1 | 2 | 3 | 4 | 5 | 6 | 7 | 8 | 9 | 10 | 11 |
| Benzyl | 2.7 | 2.7 | 2.7 | 2.7 | 2.7 | | | 50.0 | | | |
| alcohol | | | | | | | | | | | |
| PEG 400 | 44.3 | 40.3 | 49.3 | 52.3 | 7.3 | 25.0 | | | | | |
| Diisopropyl | 20.0 | | | | 15.0 | | | | | | |
| adipate | | | | | | | | | | | |
| Diethyl | | 24.0 | | | 15.0 | | | | | | |
| sebacate | | | | | | | | | | | |
| Dimethyl | | | 15.0 | | 15.0 | 25.0 | | | | | |
| isosorbide | | | | | | | | | | | |
| Propylene | | | | | | | 42.0 | | | 70.0 | 70.0 |
| glycol | | | | | | | | | | | |
| 2-(2- | 25.0 | 25.0 | 25.0 | 25.0 | 25.0 | 25.0 | | | | | |
| ethoxyethoxy) | | | | | | | | | | | |
| ethanol | | | | | | | | | | | |
| Hexylene | | | | 12.0 | 12.0 | | | | | | |
| glycol | | | | | | | | | | | |
| Oleic acid | | | | | | | 25.0 | | | | |
| Tween 80 | 8.0 | 8.0 | 8.0 | 8.0 | 8.0 | | 8.0 | | | | |
| (Polysorbate | | | | | | | | | | | |
| 80) | | | | | | | | | | | |
| Isopropyl | | | | | | | 25.0 | | | | |
| myristate | | | | | | | | | | | |
| Glycerin | | | | | | | | 50.0 | | | 30.0 |
| DMSO | | | | | | 25.0 | | | 70.0 | | |
| Water pH | | | | | | | | | 30.0 | | |
| 5 | | | | | | | | | | | |
| Phenoxy- | | | | | | | 1.0 | | | | |
| ethanol | | | | | | | | | | | |
| Water | | | | | | | | | | 30.0 | |
| Total | 100.0 | 100.0 | 100.0 | 100.0 | 100.0 | 100.0 | 101.0 | 100.0 | 100.0 | 100.0 | 100.0 |
| DMVT- | ≤1.0 | 1.3< | 1.0< | ≤1.6 | ≤1.0 | 2.3< | 1.0< | 2.0< | ≤0.5 | ≤0.5 | ≤0.5 |
| 503 | | <1.8 | <1.6 | | | <2.9 | <3.5 | <2.3 | | | |
| Solubility | | | | | | | | | | | |
| Range (%) | | | | | | | | | | | |

Mixes 1-6 and 8 were set up to analyze stability for 2 weeks at 5° C., 25° C., 30° C., and 40° C. and for 3 days at 50° C. The total impurities identified were less than 1.74% at 2 weeks under all conditions. Mixes 1, 4, and 5 were the most stable, with total impurities less than 0.5% at 2 weeks under all conditions. Table 2 provides the short-term stability data for Mix 1, Mix 4, and Mix 5.

TABLE 2
Short-Term Stability Data for Prototype Solvent Systems

| | | | | T = 3 | T = 2 | T = 2 | T = 2 | T = 2 |
|---|---|---|---|---|---|---|---|---|
| Sample | | | | days | weeks | weeks | weeks | weeks |
| Mix Composition | | Test | T(0) | (50° C.) | (5° C.) | (25° C.) | (30° C.) | (40° C.) |
| Placebo | | Assay | N/A | N/A | N/A | N/A | N/A | N/A |
| | | (% LC) | | | | | | |
| | | Impurities | N/A | N/A | N/A | N/A | N/A | N/A |
| | | (% w/w) | | | | | | |
| Mix | DMVT-503, Benzyl | Assay | 99.6 | 101.9 | 93.8 | 90.4 | 119.1 | 94.3 |
| 1 | Alcohol, PEG 400, | (% LC) | | | | | | |
| | Diisopropyl adipate, | Total | 0.15 | 0.21 | 0.13 | 0.13 | 0.23 | 0.20 |
| | 2-(2- | Impurities | | | | | | |
| | ethoxyethoxy)ethanol | (% w/w) | | | | | | |
| | (Transcutol P) Tween | | | | | | | |
| | 80 | | | | | | | |
| Mix | DMVT-503, Benzyl | Assay | 97.9 | 106.6 | 95.4 | 87.4 | 90.6 | 94.1 |
| 4 | Alcohol, PEG 400, | (% LC) | | | | | | |
| | 2-(2- | Total | 0.12 | 0.27 | 0.13 | 0.12 | 0.13 | 0.24 |
| | ethoxyethoxy)ethanol | Impurities | | | | | | |
| | (Transcutol P), | (% w/w) | | | | | | |
| | Hexylene glycol, | | | | | | | |
| | Tween 80 | | | | | | | |
| Mix | DMVT-503, Benzyl | Assay | 96.9 | 93.3 | 116.8 | 97.8 | 95.7 | 89.3 |
| 5 | Alcohol, PEG 400, | (% LC) | | | | | | |
| | Diisopropyl adipate, | Total | 0.14 | 0.91 | 0.22 | 0.28 | 0.26 | 0.50 |
| | Diethyl sebacate, | Impurities | | | | | | |
| | Dimethyl isosorbide, | (% w/w) | | | | | | |
| | 2-(2- | | | | | | | |
| | ethoxyethoxy)ethanol | | | | | | | |
| | (Transcutol P), | | | | | | | |
| | Hexylene glycol, | | | | | | | |
| | Tween 80 | | | | | | | |

### Example 2: Prototype Formulations and In Vitro Skin Penetration Study

Table 3 provides 6 topical formulations containing DMVT-503. Table 4 provides short term stability of DMVT-503 formulation prototypes.

TABLE 3
DMVT-503 Topical Formulation Prototypes
(ingredients provided as % w/w)

| | | | Formu- | Formu- | Formu- | Formu- |
|---|---|---|---|---|---|---|
| | Formu- | Formu- | lation 3 | lation 4 | lation 5 | lation 6 |
| | lation 1 | lation 2 | (SS5, | (SS5, | (G5C, | (G5C, |
| Ingredient | (SS2) | (SS4) | 974) | HPC) | HPC) | 974) |
| DMVT-503 | 0.5 | 0.6 | 0.6 | 0.6 | 0.6 | 0.6 |
| PEG 400 | 25 | 45 | 35 | 35 | | |
| Dimethyl | | | | | 15 | 15 |
| isosorbide | | | | | | |
| Propylene | 20 | | 20 | 20 | 32 | 32 |
| glycol | | | | | | |
| 2-(2- | 42.9 | 42.8 | 42.8 | 42.8 | 38.8 | 39.3 |
| ethoxyethoxy) | | | | | | |
| ethanol | | | | | | |
| (Transcutol P) | | | | | | |
| Hydroxy- | 1.5 | 1.5 | | 1.5 | 1.5 | |
| propyl | | | | | | |
| cellulose | | | | | | |
| (HPC | | | | | | |
| MXF) | | | | | | |
| Carbopol 974 | | | 1 | | | 1 |
| Glycerin | 10 | 10 | | | 12 | 12 |
| Butylated | 0.1 | 0.1 | 0.1 | 0.1 | 0.1 | 0.1 |
| hydroxy- | | | | | | |
| toluene | | | | | | |
| (BHT) | | | | | | |
| Total | 100 | 100 | 100 | 100 | 100 | 100 |
| Solubility | 6.76 | 7.20 | 7.39 | 7.39 | 7.43 | 7.34 |
| (mg/ml) | | | | | | |

TABLE 4
Short Term Stability of DMVT-503 Formulation Prototypes

| Formu- | | | T = 1 Months | | T = 2 Months | | T = 3 Months | |
|---|---|---|---|---|---|---|---|---|
| lation | | | 25° C./60 | 40° C./75 | 25° C./60 | 40° C./75 | 25° C./60 | 40° C./75 |
| Prototype | Test | T = 0 | % RH | % RH | % RH | % RH | % RH | % RH |
| SS2 | Appearance | Clear | Clear | Clear | Clear | Clear | Clear | Clear |
| | | transparent | transparent | transparent | transparent | transparent | transparent | transparent |
| | | gel | gel | gel | gel | gel | gel | gel |
| | Microscopy | Absence | Absence | Absence | Absence | Absence | Absence | Absence |
| | | of crystals | of crystals | of crystals | of crystals | of crystals | of crystals | of crystals |
| | | and no | and no | and no | and no | and no | and no | and no |
| | | birefringence | birefringence | birefringence | birefringence | birefringence | birefringence | birefringence |
| | | observed | observed | observed | observed | observed | observed | observed |
| | | under | under | under | under | under | under | under |
| | | polarizer | polarizer | polarizer | polarizer | polarizer | polarizer | polarizer |
| | Assay | 101.6 | 100.9 | 100.5 | 101.3 | 100.9 | 100.1 | 100.2 |
| | (% LC) | | | | | | | |
| | Viscosity | 13443.75 | 13562.5 | 13156.25 | 13231.25 | 12868.75 | 12812.5 | 12531.25 |
| SS4 | Appearance | Clear | Clear | Clear | Clear | Clear | Clear | Clear |
| | | transparent | transparent | transparent | transparent | transparent | transparent | transparent |
| | | gel | gel | gel | gel | gel | gel | gel |
| | Microscopy | Absence | Absence | Absence | Absence | Absence | Absence | Absence |
| | | of crystals | of crystals | of crystals | of crystals | of crystals | of crystals | of crystals |
| | | and no | and no | and no | and no | and no | and no | and no |
| | | birefringence | birefringence | birefringence | birefringence | birefringence | birefringence | birefringence |
| | | observed | observed | observed | observed | observed | observed | observed |
| | | under | under | under | under | under | under | under |
| | | polarizer | polarizer | polarizer | polarizer | polarizer | polarizer | polarizer |
| | Assay | 100.1 | 99.4 | 99.6 | 99.9 | 99.8 | 98.9 | 98.9 |
| | (% LC) | | | | | | | |
| | Viscosity | 15950 | 15881.25 | 15237.5 | 15950 | 14963.75 | 15506.25 | 14362.5 |
| SS5 | Appearance | Clear | Clear | Clear | Clear | Clear | Clear | Clear |
| (HPC) | | transparent | transparent | transparent | transparent | transparent | transparent | transparent |
| | | gel | gel | gel | gel | gel | gel | gel |
| | Microscopy | Absence | Absence | Absence | Absence | Absence | Absence | Absence |
| | | of crystals | of crystals | of crystals | of crystals | of crystals | of crystals | of crystals |
| | | and no | and no | and no | and no | and no | and no | and no |
| | | birefringence | birefringence | birefringence | birefringence | birefringence | birefringence | birefringence |
| | | observed | observed | observed | observed | observed | observed | observed |
| | | under | under | under | under | under | under | under |
| | | polarizer | polarizer | polarizer | polarizer | polarizer | polarizer | polarizer |
| | Assay | 102.3 | 101.5 | 101.5 | 102.0 | 100.9 | 101.1 | 101.0 |
| | (% LC) | | | | | | | |
| | Viscosity | 8918.75 | 10450 | 9887.5 | 9568.75 | 11118.75 | 10475 | 10393.75 |
| G5C | Appearance | Clear | Clear | Clear | Clear | Clear | Clear | Clear |
| (HPC) | | transparent | transparent | transparent | transparent | transparent | transparent | transparent |
| | | gel | gel | gel | gel | gel | gel | gel |
| | Microscopy | Absence | Absence | Absence | Absence | Absence | Absence | Absence |
| | | of crystals | of crystals | of crystals | of crystals | of crystals | of crystals | of crystals |
| | | and no | and no | and no | and no | and no | and no | and no |
| | | birefringence | birefringence | birefringence | birefringence | birefringence | birefringence | birefringence |
| | | observed | observed | observed | observed | observed | observed | observed |
| | | under | under | under | under | under | under | under |
| | | polarizer | polarizer | polarizer | polarizer | polarizer | polarizer | polarizer |
| | Assay | 101.3 | 100.4 | 100.2 | 101.0 | 100.2 | 100.3 | 99.7 |
| | (% LC) | | | | | | | |
| | Viscosity | 12031.25 | 11775 | 11593.75 | 11993.75 | 11968.75 | 11518.75 | 11043.75 |

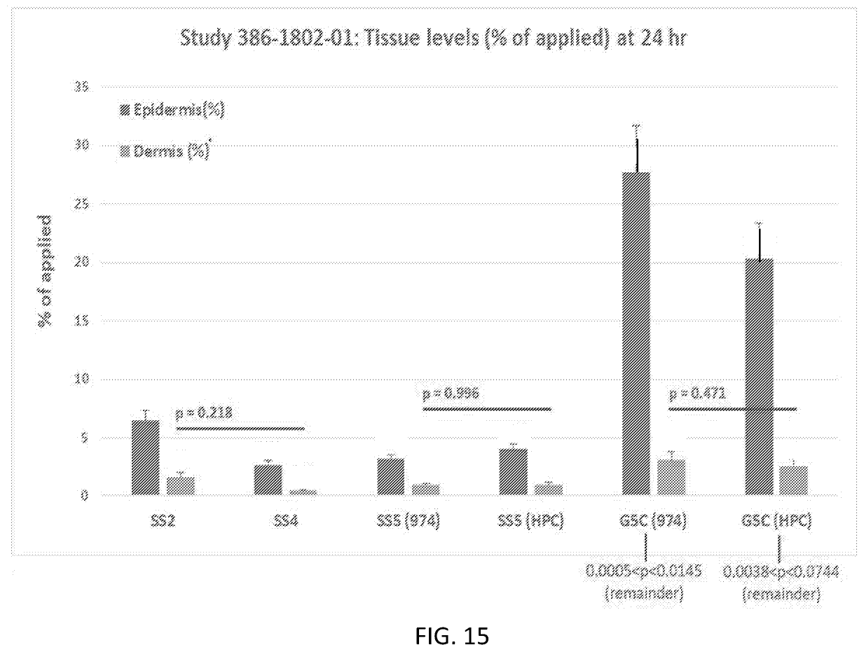

G5C formulations with either hydroxypropylcellulose (HPC MXF) or Carbopol 974 as the gelling agents delivered more drug into the skin tissue at ˜89× the IC₅₀ values. FIG. 15 provides the percentage of drug detected within the epidermis or dermis after 24 hours of application (3 human skin donors, n=15). Table 5 provides the data as shown in the graph of FIG. 15.

TABLE 5
Tissue levels (% of applied) at 24 hours

| Formulation | Epidermis (%) | Dermis (%) |
|---|---|---|
| SS2 | 6.49 | 1.59 |
| SS4 | 2.64 | 0.45 |
| SSS (974) | 3.16 | 0.97 |
| SSS (HPC) | 9.38 | 0.97 |
| GSC (974) | 22.70 | 3.06 |
| GSC (HPC) | 20.32 | 2.56 |

### Example 3: Mechanism of Action Pharmacology Studies

Acne and seborrhoea are sebaceous gland-related diseases are exclusively human diseases. Accordingly, in vitro research is performed on human sebocyte culture. Cultured human sebocytes have been shown to preserve important sebocytic characteristics, and have become a useful tool in studying sebaceous gland activity and regulation, and understanding the pathophysiological mechanisms and treatment of acne and other sebaceous gland related diseases.

Three assays have been performed using human sebocyte cell lines: Nile Red Neutral Lipid Accumulation Assay, 14C-Acetate Sebocyte Lipid Accumulation Assay, and Cleaved Caspase 3 Assay.

A. Nile Red Neutral Lipid Accumulation Assay

This assay examines the ability of compounds to stimulate or inhibit lipid accumulation in human sebocyte cell line. Cells were seeded in tissue culture treated black clear bottom plates. Compounds were added in the presence of Liver X Receptor (LXR) agonist (T0901317) and 1 μM insulin for 3 days and 5 days. For the 5 day treatment fresh compounds were added at Day 3. After the treatment period nile red dye was added and the relative fluorescence determined for both total and neutral lipids. The neutral/total lipid ratio was calculated and obtained values were normalized to both the Cell Titer Blue viability reagent.

Protocol—Day 1: Human immortalized sebocytes were seeded at 10,000 cells/well in 96-well black, clear bottom plates in Sebocyte Growth Medium and allowed to attach overnight at 37° C. Day 2: Medium was refreshed by removing seeding medium and adding fresh Sebocyte Growth Medium. Cells were maintained at 37° C. to allow degradation of the temperature sensitive SV40 large T antigen. Day 4: 1000× stocks (in DMSO) of each compound were prepared (3 mM and 100 μM) and were diluted 1:1000 in Sebocyte Treatment Media containing 1 μM T0901317 and 1 μM Insulin to give a final top concentration of 3000 nM and 100 nM for further 10-fold dilutions in the same medium according to the plate map below. The cell medium was removed and replaced with Sebocyte Treatment Medium containing 1 μM T0901317, 1 μM Insulin, compound dilution, 2 μM A922500 or vehicle (DMSO) as per plate maps for a 3 day and 5 day treatment. For untreated controls, media was removed and replaced with Sebocyte Treatment Media only (no T091317 or Insulin). For the 5 day treatment cells were retreated as described above on Day 7. Day 7 or Day 9: 1) All treatment medium was replaced with 100 μl of Nile Red in PBS at a concentration of 1 μg/ml and the cells were allowed to incubate for 30 minutes at 37° C., 5% CO2. 2) After 30 minutes, the Nile red was removed and replaced with 50 μl of PBS. 3) The relative fluorescence (RFU) of the incorporated stain was determined under 2 parameters: 540ₑₓ/620ₑₘ with no cutoff (Total Lipid), and 485ₑₓ/555ₑₘ with a 515 cutoff (Neutral Lipid) using bottom reads. 4) Following lipid analysis, PBS was removed and 50 μl of sebocyte treatment medium (no T091317 or Insulin) was added to each well. Cell titer blue reagent (100 was then added to each well and the cells were allowed to incubate for 2 hours at 37° C., 5% CO2. 5) The RFU value was determined at 560ₑₓ/590ₑₘ with a 570 cutoff using a top read.

Data Analysis—Cell viability is presented as cell titer blue fluorescence reading (RFU) for each treatment. A decrease in CTB RFU represents a decrease in cell viability. For lipid analysis neutral lipid RFU were normalized to total lipid RFU values in each well. Neutral and total lipid RFU values were normalized to CTB RFU in each well. Normalized values for each treatment were averaged and presented relative to controls. IC50s were determined for each test sample by using appropriate curve fits in GraphPad Prism (v.7). Minimum and maximum values are determined by UNT and LXR+INS controls.

Treatment groups: Untreated (− insulin), Vehicle (+ insulin and DMSO), Stimulated (+insulin+1 μM LXR agonist T0901317), Inhibited (+insulin+2 μM DGAT Inhibitor A922500), DMVT-503, and 13-cis-retinoic Acid (comparator).

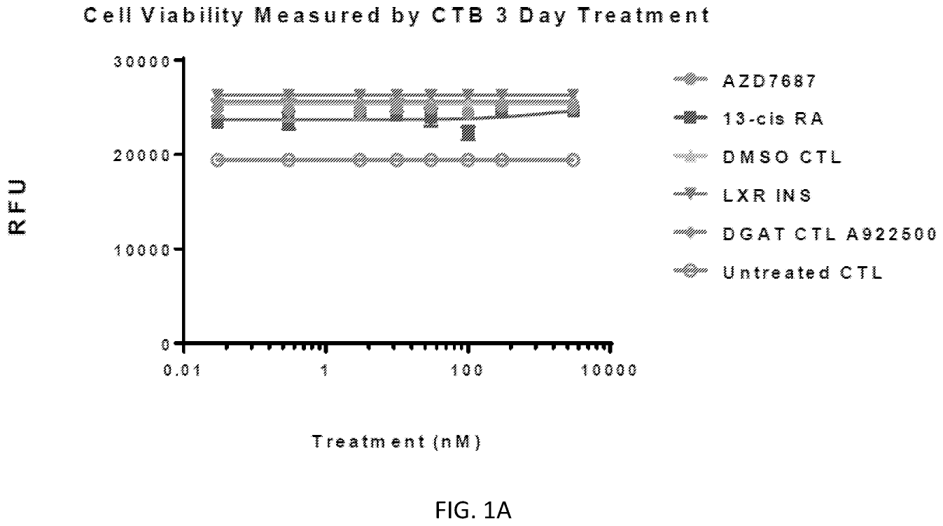

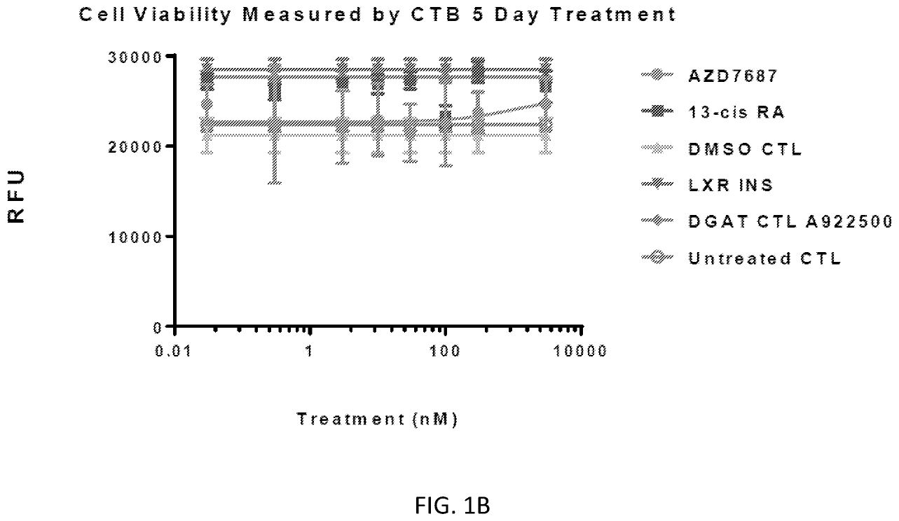

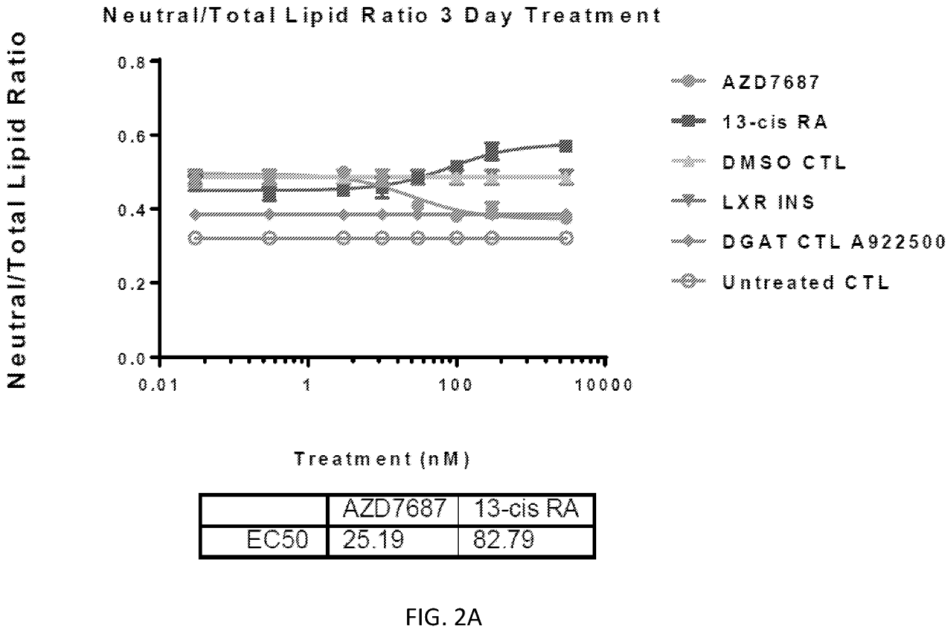

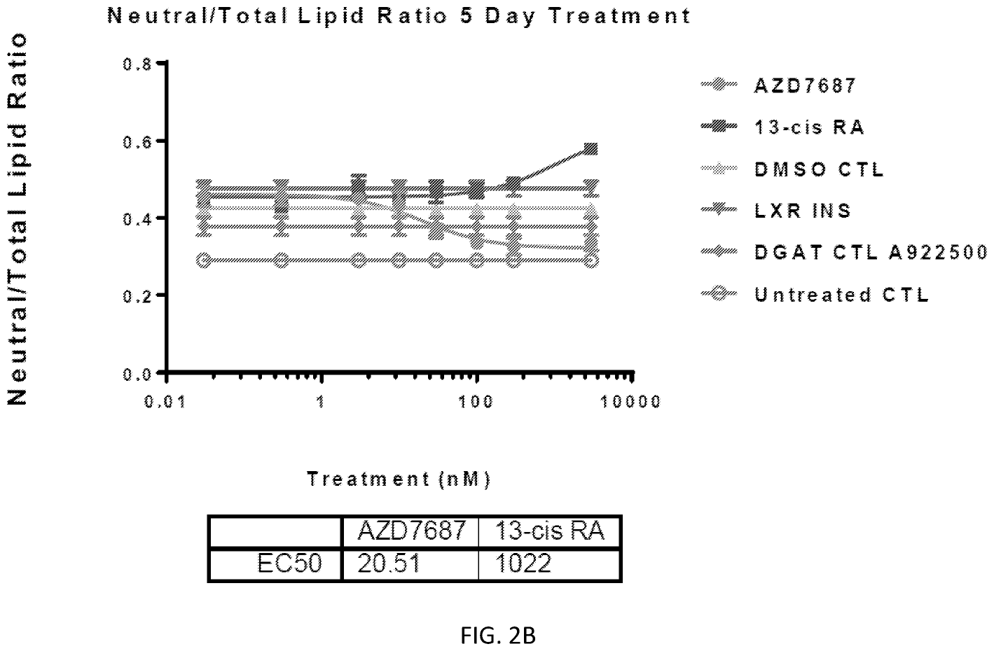

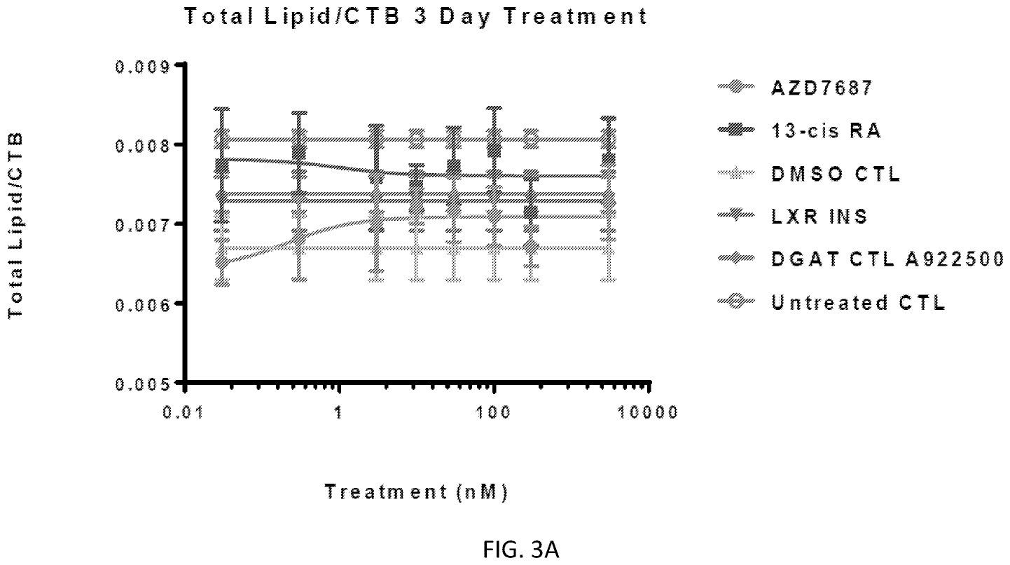

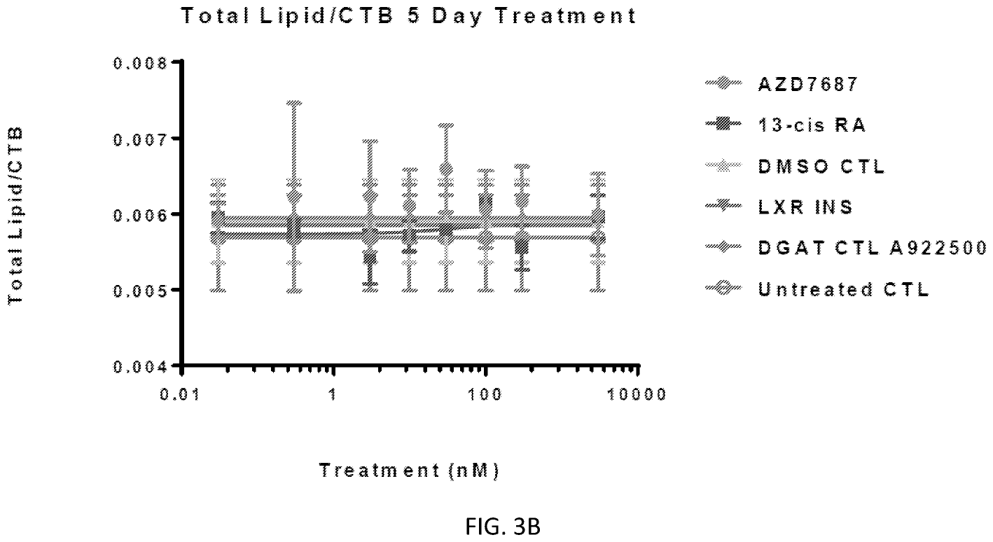

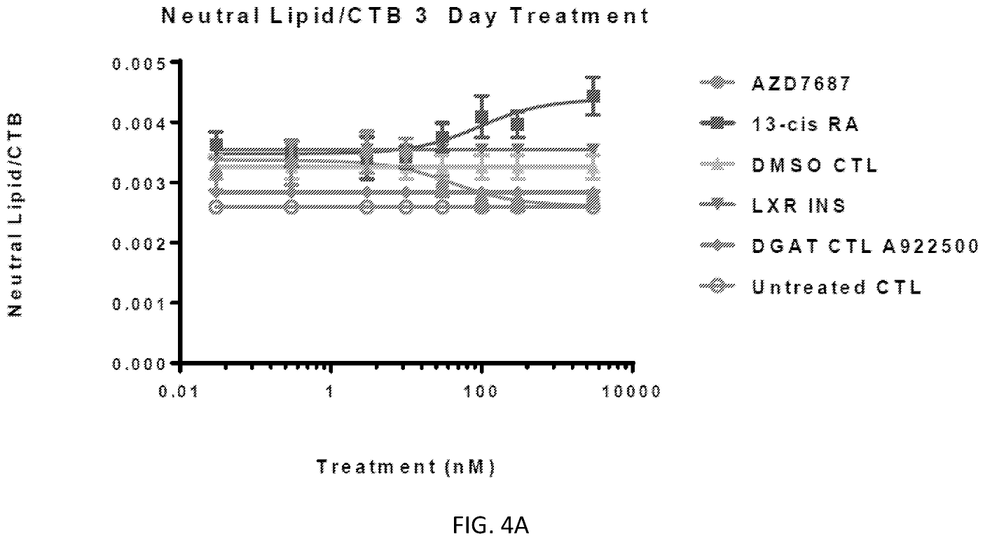

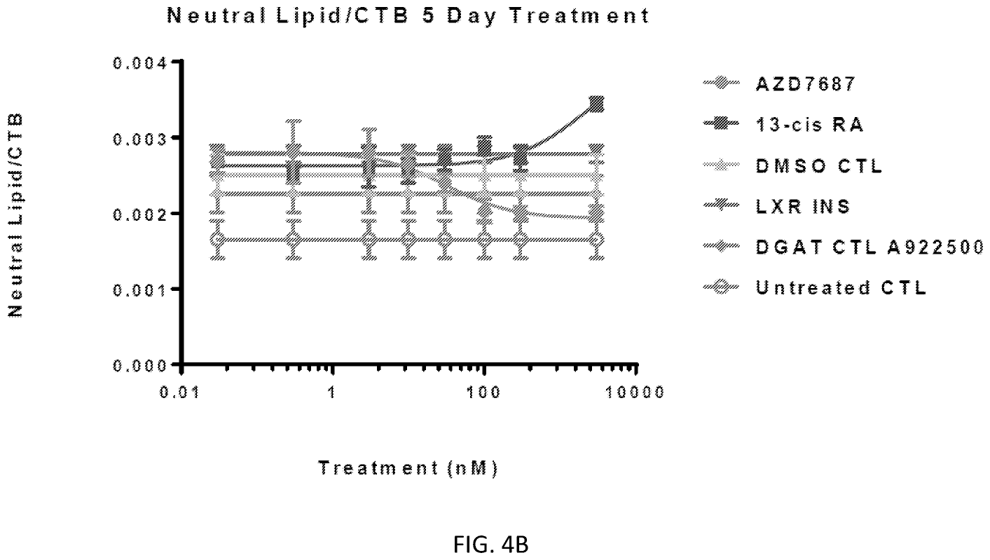

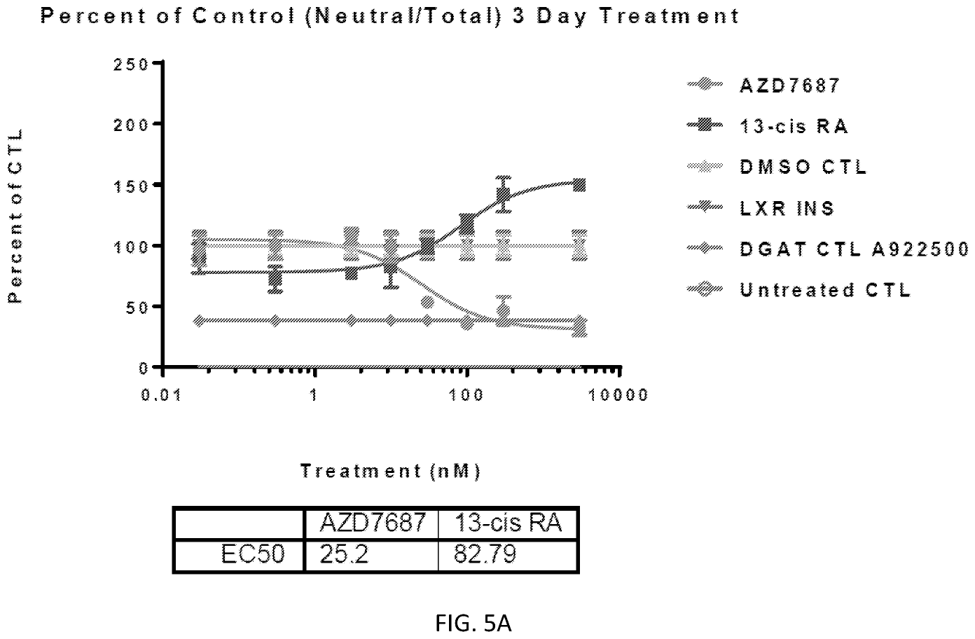

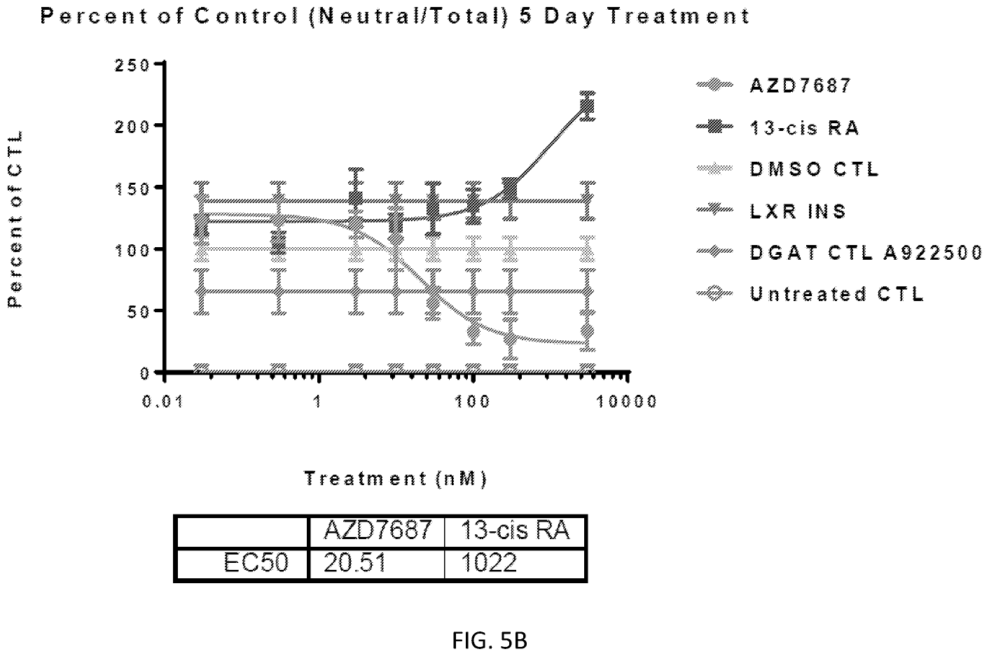

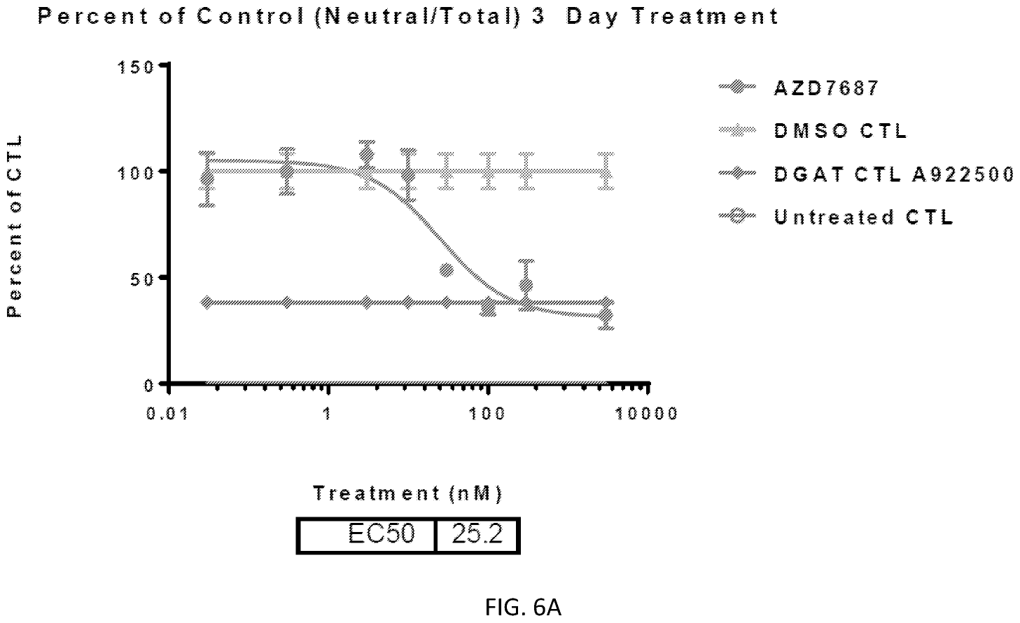

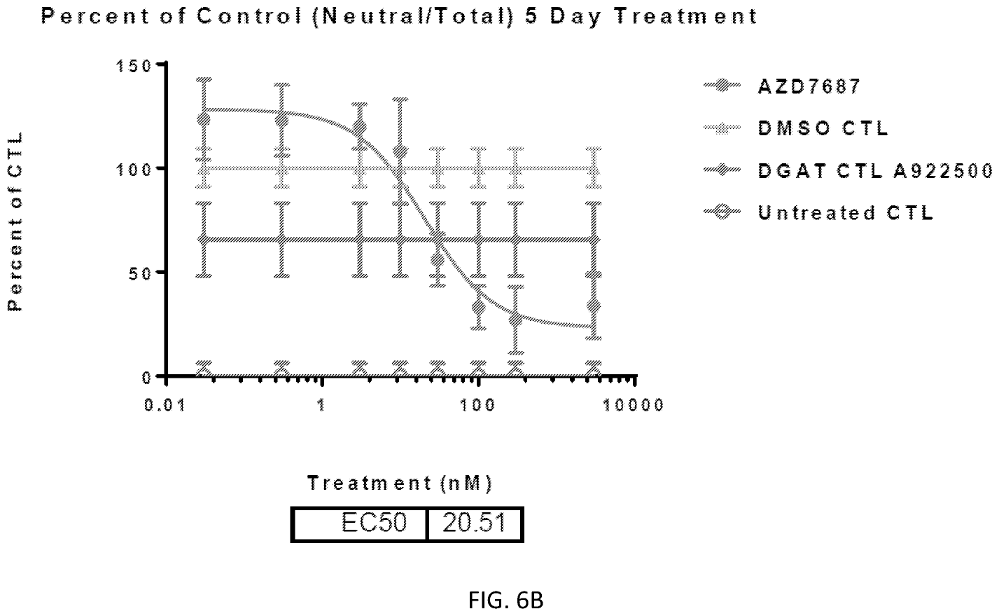

Results—FIG. 1A provides cell viability data from day 3 and FIG. 1B provides cell viability data from day 5. FIG. 2A provides neutral/total lipid ratio data from day 3 and FIG. 2B provides neutral/total lipid ratio data from day 5. FIG. 3A provides total lipid normalized to CTB signal at day 3 and FIG. 3B provides total lipid normalized to CTB signal at day 5. FIG. 4A provides neutral lipid normalized to CTB signal at day 3 and FIG. 4B provides neutral lipid normalized to CTB signal at day 5. FIG. 5A provides percent of control (neutral/total) at day 3 and FIG. 5B provides percent of control (neutral/total) at day 5. FIG. 6A provides the DMVT-503 dose response curve as a percent of control on day 3 and FIG. 6B provides the DMVT-503 dose response curve as a percent of control on day 5. DMVT-503 day 3 IC₅₀ was 25.2 nM whereas 13-cis-RA was 828.8 nM.

B. 14C-Acetate Sebocyte Lipid Accumulation Assay

This assay examines the ability of compounds to modulate the incorporation of label into de novo lipid synthesis. Cells were seeded in collagen 1-coated Scintiplates. Compounds were added in the presence of Liver X Receptor (LXR) agonist (T0901317) overnight. The medium was replaced with treatments and stimulated with 1 μM insulin for 4 hours in Labeling Medium containing 14C-acetate. Label incorporation was determined by washing the cells to removed excess label and allowing the cell layer to air dry before assessing labeling through Scintiplate counting normalized to Cell Titer Blue viability reagent.

Protocol—Day 1: Human immortalized sebocytes were seeded at 10,000 cells/well in a 96-well white, clear bottom collagen 1-coated Scintiplate in Sebocyte Growth Medium and allowed to attach overnight at 37° C. Day 2: Medium was refreshed by removing seeding medium and adding fresh Sebocyte Growth Medium. Cells were maintained at 37° C. to allow degradation of the temperature sensitive SV40 large T antigen. Day 4: 1000× stocks (in DMSO) of each compound were prepared (3 mM and 100 μM) and were diluted 1:1000 in Sebocyte Labeling Media containing 1 μM T0901317 to give a final top concentration of 3000 nM and 100 nM for further 10-fold dilutions in the same medium according to the platemap below. The cell medium was removed and replaced with Sebocyte Labeling Medium containing 1 μM T0901317, compound dilution, 2 μM A922500 or vehicle (DMSO) as per plate maps for a 16 hour (overnight) treatment. For untreated controls, media was removed and replaced with Sebocyte Labeling Media only (no T091317). Day 5: 1) All treatment medium was replaced with Sebocyte Labeling Medium containing 1 μM T0901317 and original treatments or vehicle (DMSO) plus 104 insulin. The untreated control was replaced with Sebocyte Labeling Media only. 1 μl of 14C-acetate was added per well (in total volume of 10 μl with Sebocyte Labeling Medium) and the cells incubated for 4 hours at 37° C. 2) After 2 hours of incubation, 10 μl of CTB reagent was added per well for normalization. Incubation was continued for the remaining 2 hours. 3) Plates were read at Ex. 560 Em.590 with a cutoff of 570 nm for CTB signal. 4) Cells were then washed three times with PBS and the cell layer allowed to air dry. 5) 14C-acetate labeling was determined by scintillation counting in a MicroBeta TriLux Scintillation Counter.

Data analysis—Cell viability is presented as cell titer blue fluorescence reading (RFU) for each treatment. A decrease in CTB RFU represents a decrease in cell viability. The percent of LXR agonist+Insulin-stimulated lipogenesis was calculated by taking the ratio of each CPM measure over the corresponding cell titer blue measure. This ratio was normalized by subtracting the untreated control ratio from each ratio value. The resulting normalized ratio was then divided by that of the normalized LXR+INS stimulated control and multiplied by 100. The resulting number yields the percent of LXR agonist+insulin-stimulated lipogenesis.

Treatment groups: Untreated (UNT—Labeling Medium for 16 hours), VEH (Labeling Medium+1 μM T0901317+0.1% DMSO for 16 hours+1 μM insulin for 4 hours), LXR+INS (Labeling Medium+1 μM T0901317 for 16 hours+1 μM insulin for 4 hours), A922500 (Labeling Medium+1 μM T0901317+204 A922500 for 16 hours+1 μM insulin for 4 hours), and Compounds (Labeling Medium+1 μM T0901317+Compounds for 16 hours+1 μM insulin for 4 hours).

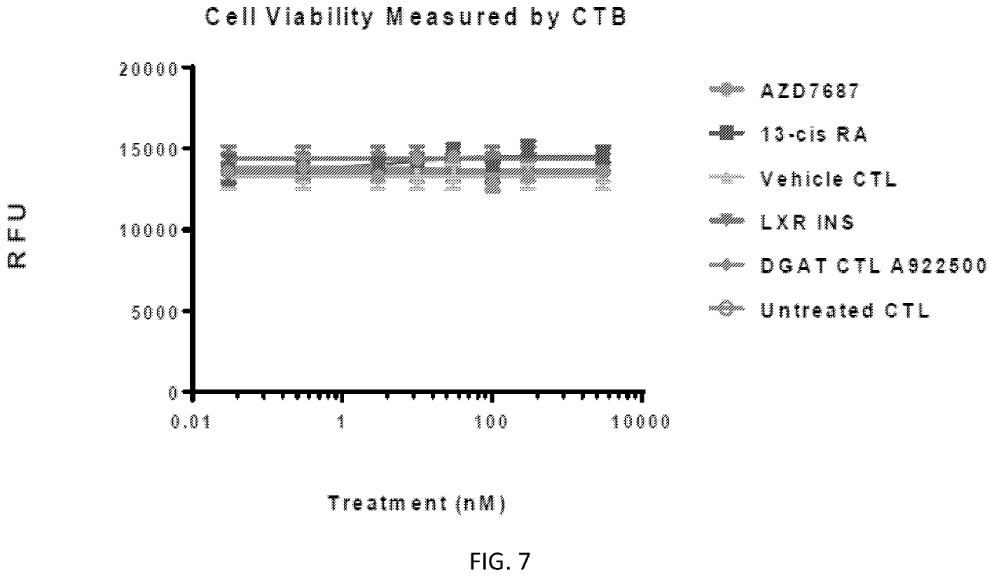

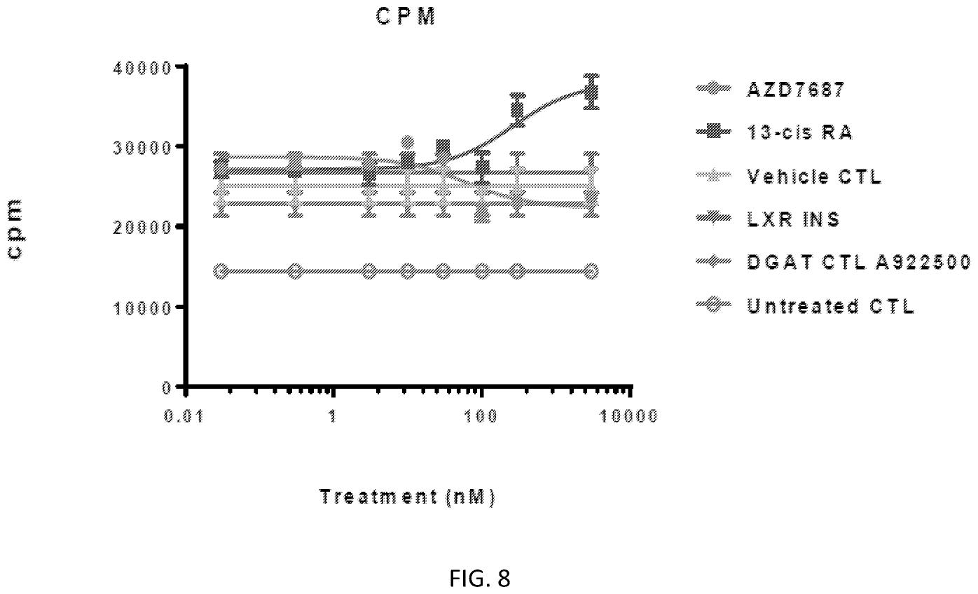

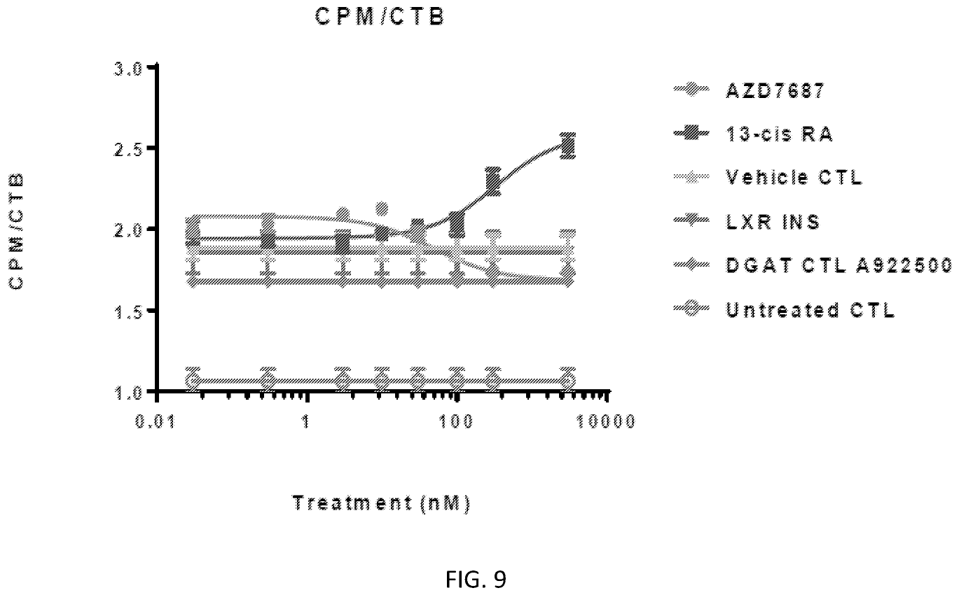

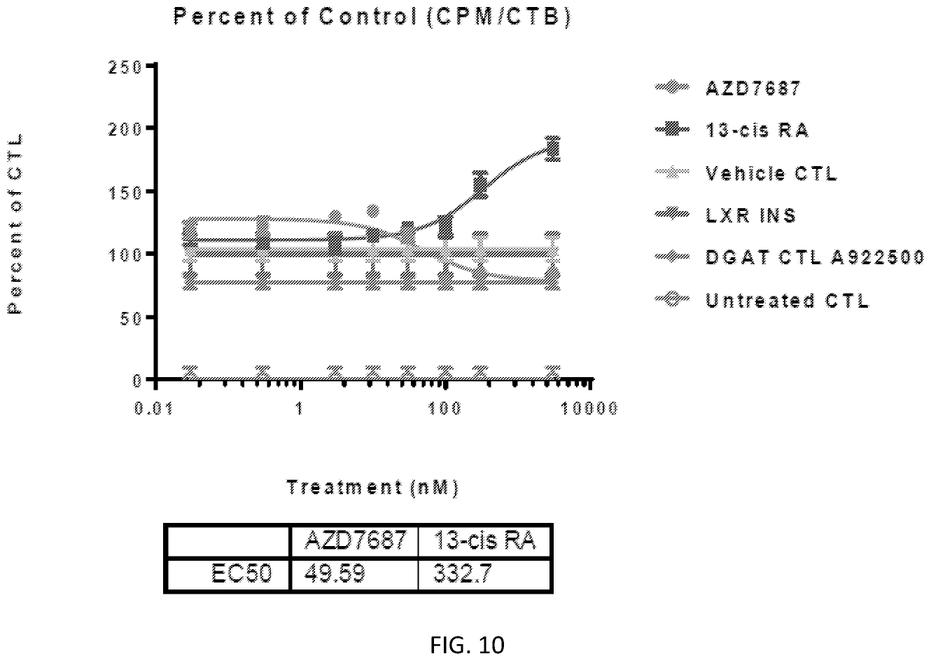

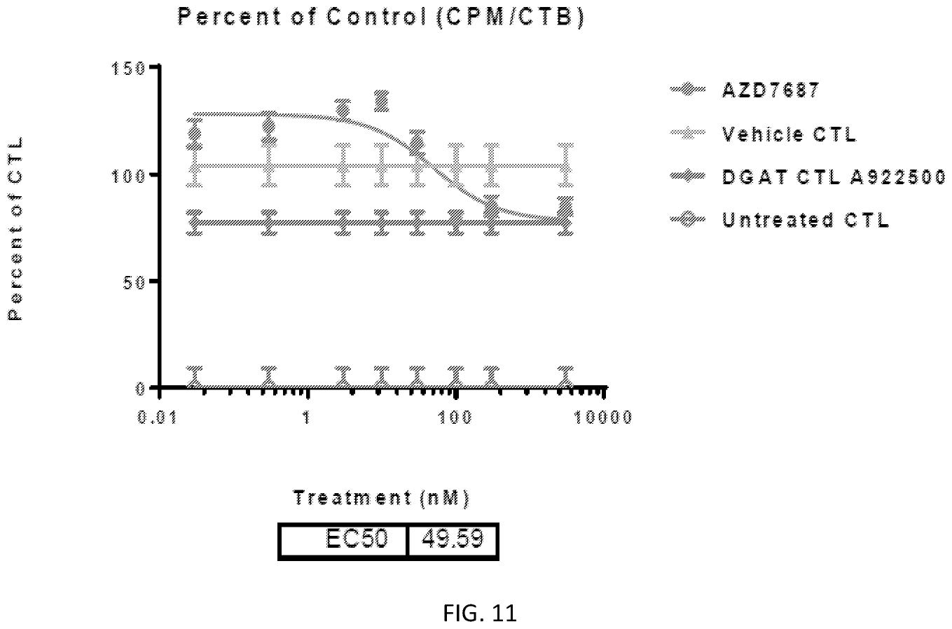

Results—FIG. 7 provides cell viability data measured by CTB. FIG. 8 provides the raw CPM data. FIG. 9 provides CPM data normalized to CTB signal. FIG. 10 provides the percent of positive control (LXR+INS). FIG. 11 provides the DMVT-503 dose response curve as a percent of positive control (LXR+INS). DMVT-503 day 2 IC₅₀ was 49.6 nM whereas 13-cis-RA was 332.7 nM.

C. Cleaved Caspase 3 Assay

This assay examines the ability of compounds to induce apoptosis by measuring Caspase 3 generated by proteolytic processing. Cells were seeded in tissue culture treated clear bottom plates. Compounds were added in the presence of Liver X Receptor (LXR) agonist (T0901317) and 1 μM insulin for 2, 3 and 5 days. For the 5 day treatment fresh compounds were added at Day 3. After the treatment period lysates were prepared according to the manufacturer's instructions and frozen at −80° C. until the day of the assay.

Protocol—Day 1: Human immortalized sebocytes were seeded at 10,000 cells/well in 96-well, clear bottom plates in Sebocyte Growth Medium and allowed to attach overnight at 37° C. Day 2: Medium was refreshed by removing seeding medium and adding fresh Sebocyte Growth Medium. Cells were maintained at 37° C. to allow degradation of the temperature sensitive SV40 large T antigen. Day 4: 1000× stocks (in DMSO) of each compound were prepared (3 mM and 100 μM) and were diluted 1:1000 in Sebocyte Treatment Media containing 1 μM T0901317 and 1 μM Insulin to give a final top concentration of 3000 nM and 100 nM for further 10-fold dilutions in the same medium according to the plate map below. The cell medium was removed and replaced with Sebocyte Treatment Medium containing 1 μM T0901317, 1 μM Insulin, compound dilution, or vehicle (DMSO) as per plate maps for a 2, 3 and 5 day treatment. For untreated controls, media was removed and replaced with Sebocyte Treatment Media only (no T091317 or Insulin). For the 5 day treatment cells were retreated as described above on Day 7. Day 6, 7 or Day 9: 1) After treatment, the plate was centrifuged at 300×g for 10 minutes. 2) After centrifugation, the medium was removed the cells rinsed with ice-cold PBS and then centrifuged again at 300×g for 10 minutes. 3) After centrifugation the PBS was removed and replaced with 30 μl/well and left on ice for 5 minutes. 4) After 5 minutes, the lysates were frozen at −80° C. until the day of the assay. Day of Assay: Buffers and reagents were prepared according to the manufacturer's instructions. 1) 25 μl of lysate was added to the corresponding well of a 96 well black clear bottom plates. 2) 200 μl of the prepared substrate solution B was added to each well. 3) After 1 hr incubation in the dark, the relative fluorescence was determined using the following settings: 380ₑₓ/420ₑₘ.

Data analysis—IC50s were determined for each test sample by using appropriate curve fits in GraphPad Prism (v.7).

Treatment groups: Untreated (UNT—Treatment Medium (no LXR or Insulin) alone for 2, 3 and 5 days), VEH (Treatment Medium+1 μM T0901317+0.1% DMSO+1 μM insulin for 2, 3 and 5 days), LXR+INS (Treatment Medium+1 μM T0901317+1 μM insulin for 2, 3 and 5 days), Compounds (Treatment Medium+1 μM T0901317+Compounds+1 μM insulin for 2, 3 and 5 days).

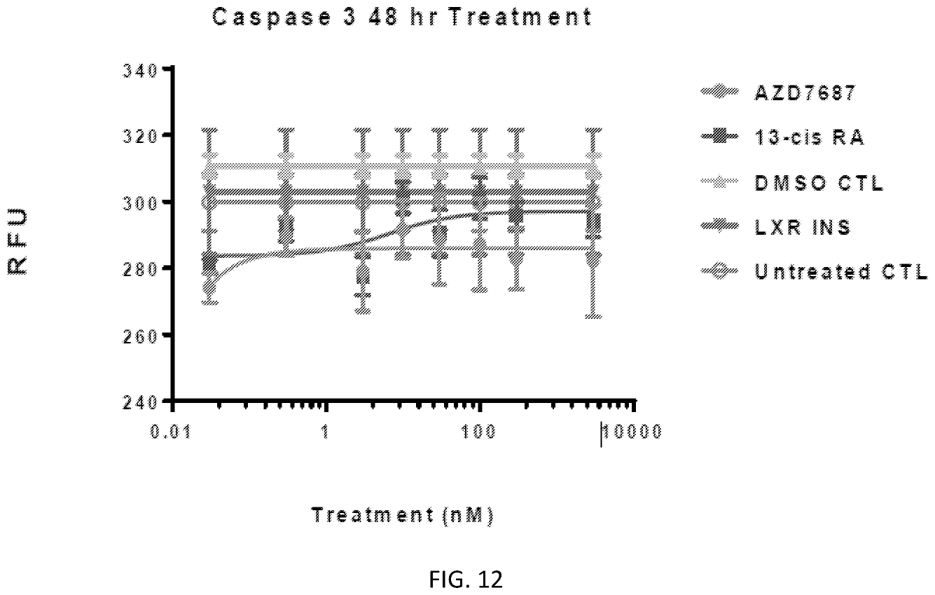

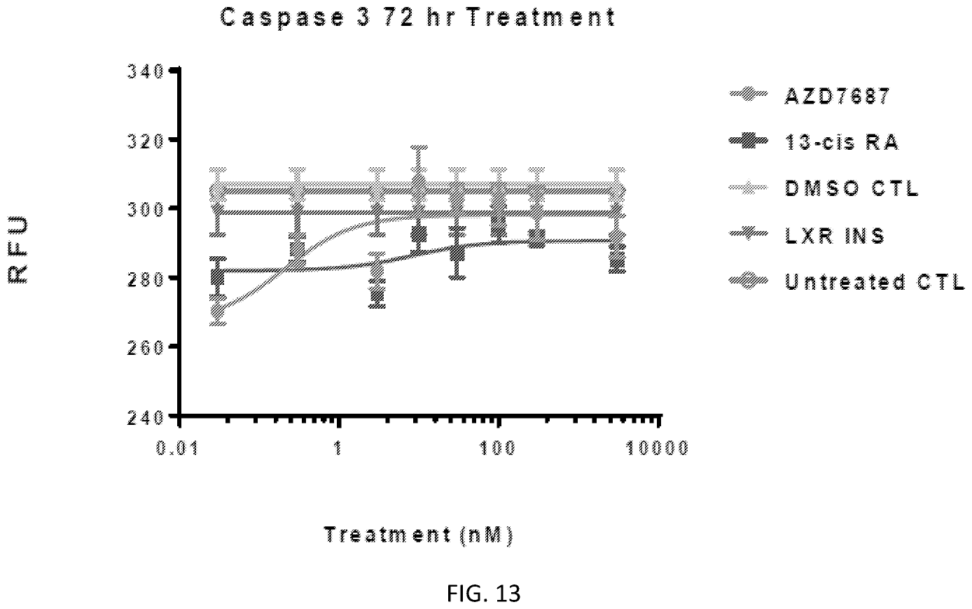

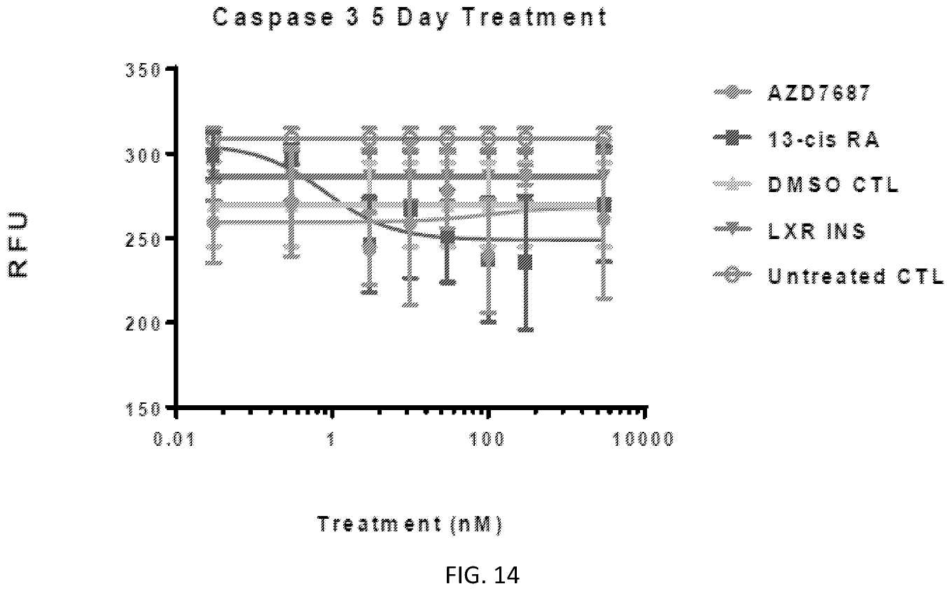

Results—FIG. 12 provides Caspase 3 data after 48 hour treatment. FIG. 13 provides Caspase 3 data after 72 hour treatment. FIG. 14 provides Caspase 3 data after 5 day treatment. Minimal changes in cell viability over 5 day incubation. No significant cleaved caspase 3 detected with any concentration of: DMVT-503 or 13-cis-RA.

Conclusions: DMVT-503 and 13-cis-RA did not induce sebocyte apoptosis under the conditions of this in vitro assay.

### Example 4: Metabolite Profiling of DMVT-503 in rat, Minipig and Human Hepatocytes

A potential human specific metabolite (amide hydrolysis product; M6) was noted in previous studies. Specifically, the formation of M6 by hydrolysis of the terminal amide to the corresponding acid. The metabolite profile of DMVT-503 was evaluated by high resolution mass spectrometry following in vitro incubations with hepatocytes from rats, minipigs and humans.

Methods: Characterization of DMVT-503 metabolites in hepatocytes-DMVT-503 (10 μM, final concentration) was incubated with pooled cryopreserved rat, minipig, and human hepatocytes (1 million cells/mL) in OptiIncubate Hepatocyte Media. DMVT-503 was added to the incubation mixtures in DMSO (0.1% v/v). Reactions were initiated by placing incubations in an incubator. The incubations were not agitated. The incubation temperature was 37±1° C., and the incubation atmosphere was a 95:5 mixture of air and CO2, 95% relative humidity. After 0, 60, 120 and 240 min of incubation, the reactions were terminated by the addition of an equal volume of acetonitrile with 2% formic acid. The samples were centrifuged (920×g for 10 min at 10° C.). The supernatant fractions were analyzed by liquid chromatography tandem mass spectrometry (LC-MS/MS) with in-line UV detection to characterize the metabolites formed from DMVT-503. Incubations of DMVT-503 for 0 and 240 min with boiled (denatured) hepatocytes were used as negative controls to distinguish metabolites from chemical degradation products. 7-Ethoxycoumarin (500 μM, final concentration) was used as a positive control substrate to evaluate the metabolic competency of the hepatocytes.

Analytical experimental procedures for metabolite profiling and characterization—All samples were analyzed by ultra-high performance liquid chromatography tandem mass spectrometry (UHPLC-MS/MS) with a quadrupole time of flight mass spectrometer, for acquisition of high-resolution accurate mass data, in positive mode with electrospray ionization incorporating in-line UV detection with a photodiode array detector.

Table 6 provides the data of the conversion of 7-ethoxycoumarin (500 μM, 125,000 pmol incubated) to 7-hydroxycoumarin, 7-hydroxycoumarin sulfate and 7-hydroxycoumarin glucuronide over 60 min incubation with rat, minipig, and human hepatocytes (1 million cells/mL).

TABLE 6
Conversion of 7-ethoxycoumarin

| | 7-Hydroxycoumarin | 7-Hydroxycoumarin | 7-Hydroxycoumarin | Total metabolite |
|---|---|---|---|---|
| | formed per | glucuronide formed | sulfate formed per | detected |
| Species | incubation (pmol) | per incubation (pmol) | incubation (pmol) | (pmol) |
| Rat | 694 | 2730 | 1380 | 4800 |
| Minipig | 3170 | 4010 | 177 | 7350 |
| Human | 406 | 833 | 166 | 1410 |

In the current study, M6 (C6 in the Table 7) was formed in all 3 species (human, minipig and rat). The formation of metabolite C5 (amide hydrolysis+glucuronidation) was only noted in human hepatocyte incubations but not in incubations with rat or minipig hepatocytes. This is potentially due to the lesser formation of C6 (amide hydrolysis) in rat and minipig hepatocytes which may be a precursor step to the formation of C5 (a secondary metabolite). Table 6 shows the metabolite profiling and characterization data for DMVT-503 (10 μM) incubated for up to 240 min with rat, minipig and human hepatocytes (1 million cells/mL).

TABLE 7
Metabolite Profiling

| | Retention | Experi- | | Mass | | | Proposed | | | |
|---|---|---|---|---|---|---|---|---|---|---|
| Com- | time | mental | Theoretical | error | Mass | Elemental | biotrans- | | Mini- | |
| ponent | (min) | m/z | m/z | (ppm) | shift | composition | formation | Rat | pig | Human |
| C1 | 8.33 | 475.2008 | 475.2015 | −1.5 | 107.0034 | C₂₃H₃₀N₄O₅S | Taurine conjugation | + | ND | ND |
| C2 | 8.40 | 384.1913 | 384.1923 | −2.6 | 15.9939 | C₂₁H₂₅N₃O₄ | Hydroxylation | | +^{t} | ND |
| C3 | 9.24 | 544.2294 | 544.2295 | −0.2 | 176.0320 | C₂₇H₃₃N₃O₉ | Glucuronidation | + | + | + |
| C4 | 9.32 | 425.2181 | 425.2189 | −1.9 | 57.0207 | C₂₃H₂₈N₄O₄ | Glycine conjugation | ND | + | ND |
| C5 | 9.51 | 545.2126 | 545.2135 | −1.7 | 177.0152 | C₂₇H₃₂N₂O₁₀ | Amide hydrolysis + | ND | ND | + |
| | | | | | | | glucuronidation | | | |
| DMVT- | 11.23 | 368.1973 | 368.1974 | −0.3 | — | C₂₁H₂₅N₃O₃ | Parent | + | + | + |
| 503 | | | | | | | | | | |
| C6 | 11.60 | 369.1810 | 369.1814 | −1.1 | 0.9836 | C₂₁H₂₄N₂O₄ | Amide hydrolysis | + | + | + |

+ = Detected
ND = Not detected
T = Detectcd at trace levels (with S/N ≤ 3)

Table 8 shows the viability of hepatocytes in the absence of DMVT-503 before and during incubation. Viability was determined as a single determination by Trypan blue exclusion analysis.

TABLE 8
Cell Viability

| | Percent viability at 37 ± 1° C. | | | | |
|---|---|---|---|---|---|
| Species | Initial | 0 min | 60 min | 120 min | 240 min |
| Rat | 80.4 | 76.0 | 68.3 | 64.4 | 52.7 |
| Minipig | 79.1 | 78.4 | 64.5 | 58.3 | 50.0 |
| Human | 84.0 | 70.0 | 62.5 | 63.8 | 59.2 |

### Example 5: Evaluating Topical Bioavailability

Dermatopharmacokinetic (DPK) Studies

The dermatopharmacokinetic (DPK) approach is comparable to a blood, plasma, urine PK approach applied to the stratum corneum. DPK encompasses drug concentration measurements with respect to time and provides information on drug uptake, apparent steady-state levels, and drug elimination from the stratum corneum based on a stratum corneum concentration-time curve.

For antiacne drug products, target sites are the hair follicles and sebaceous glands. In this setting, the drug diffuses through the stratum corneum, epidermis, and dermis to reach the site of action. The drug may also follow follicular pathways to reach the sites of action. The extent of follicular penetration depends on the particle size of the active ingredient if it is in the form of a suspension. Under these circumstances, the DPK approach is still expected to be applicable because studies indicate a positive correlation between the stratum corneum and follicular concentrations.

Application and Removal of Test and Reference Products: The treatment areas are marked using a template without disturbing or injuring the stratum corneum/skin. The size of the treatment area will depend on multiple factors including drug strength, analytical sensitivity, the extent of drug diffusion, and exposure time. The stratum corneum is highly sensitive to certain environmental factors. To avoid bias and to remain within the limits of experimental convenience and accuracy, the treatment sites and arms should be randomized. Uptake, steady-state, and elimination phases, as described in more detail below, may be randomized between the right and left arms in a subject. Exposure time points in each phase may be randomized among various sites on each arm. The test and reference products for a particular exposure time point may be applied on sites to minimize differences. Test and reference products should be applied concurrently on the same subjects according to a SOP that has been previously developed and validated. The pre-marked sites are treated with predetermined amounts of the products (e.g., 5 mg/sq cm) and covered with a nonocclusive guard. Occlusion is used only if recommended in product labeling. Removal of the drug product is performed according to SOPs at the designated time points, using multiple cotton swabs or Q-tips with care to avoid stratum corneum damage. In case of certain oily preparations such as ointments, washing the area with a mild soap may be needed before skin stripping. If washing is carried out, it should be part of an SOP.

Sites and Duration of Application: The BA/BE study should include measurements of drug uptake into the stratum corneum and drug elimination from skin. Each of these elements is important to establish bioavailability and/or bioequivalence of two products, and each may be affected by the excipients present in the product. A minimum of eight sites should be employed to assess uptake/elimination from each product. The time to reach steady state in the stratum corneum should be used to determine timing of samples. For example, if the drug reaches steady-state in three hours, 0.25, 0.5, 1 and 3 hours posttreatment may be selected to determine uptake and 4, 6, 8 and 24 hours may be used to assess elimination. A zero time point (control site away from test sites) on each subject should be selected to provide baseline data. If the test/reference drug products are studied on both forearms, randomly selected sites on one arm may be designated to measure drug uptake/steady-state. Sites on the contralateral arm may then be designated to measure drug elimination. During drug uptake, both the excess drug removal and stratum corneum stripping times are the same so that the stratum corneum stripping immediately follows the removal of the excess drug. In the elimination phase, the excess drug is removed from the sites at the steady-state time point, and the stratum corneum is harvested at succeeding times over 24 hours to provide an estimate of an elimination phase.

Collection of Sample: Skin stripping proceeds first with the removal of the first 1-2 layers of stratum corneum with two adhesive tapes strip/disc applications, using a commercially available product (e.g., D-Squame, Transpore®). These first two tape-strip(s) contain the generally unabsorbed, as opposed to penetrated or absorbed, drug and therefore should be analyzed separately from the rest of the tape-strips. The remaining stratum corneum layers from each site are stripped at the designated time intervals. This is achieved by stripping the site with an additional 10 adhesive tape-strips. All ten tape strips obtained from a given time point are combined and extracted, with drug content determined using a validated analytical method. The values are generally expressed as amounts/area (e.g., ng/cm²) to maintain uniformity in reported values. Data may be computed to obtain full drug concentration-time profiles, Cₘₐₓ₋ₛₛ, Tₘₐₓ₋ₛₛ, and AUCs for the test and reference products.

Procedure for Skin Stripping:

To assess drug uptake: Apply the test and/or reference drug products concurrently at multiple sites. After an appropriate interval, remove the excess drug from a specific site by wiping three times lightly with a tissue or cotton swab. Using information from the pilot study, determine the appropriate times of sample collection to assess drug uptake. Repeat the application of adhesive tape two times, using uniform pressure, discarding these first two tape strips. Continue stripping at the same site to collect ten more stratum corneum samples. Care should be taken to avoid contamination with other sites. Repeat the procedure for each site at other designated time points. Extract the drug from the combined ten skin strippings and determine the concentration using a validated analytical method. Express the results as amount of drug per square cm treatment area of the adhesive tape.

To assess drug elimination: Apply the test and reference drug product concurrently at multiple sites chosen based on the results of the pilot study. Allow sufficient exposure period to reach apparent steady-state level. Remove any excess drug from the skin surface as described previously, including the first two skin strippings. Collect skin stripping samples using ten successive tape strips at time intervals based on the pilot study and analyze them for drug content.

Metrics and Statistical Analyses: A plot of stratum corneum drug concentration versus a time profile should be constructed to yield stratum corneum metrics of Cₘₐₓ, Tₘₐₓ and AUC. The two one-sided hypotheses at the α=0.05 level of significance should be tested for AUC and Cₘₐₓ by constructing the 90 percent confidence interval (CI) for the ratio between the test and reference averages. Individual subject parameters, as well as summary statistics (average, standard deviation, coefficient of variation, 90% CI) should be reported. For the test product to be, the 90 percent CI for the ratio of means (population geometric means based on log-transformed data) of test and reference treatments should fall within 80-125 percent for AUC and 70-143 percent for Cₘₐₓ.

In Vivo Dermal Open Flow Microperfusion

In dermal open-flow microperfusion (dOFM), a thin, hollow tube is inserted just under the skin surface, running through a section of the skin a few inches wide and then exiting. A liquid similar to body fluid is injected into the tubing; a portion of the tube under the skin is porous, so any drug that has been applied and absorbed through the skin's outer layer enters the flowing liquid, which is then collected for analysis. dOFM can reliably measure the changing amounts of drug in the skin after topical application of a dermatological drug product.

### Example 6: Ex Vivo Skin Permeation and Penetration

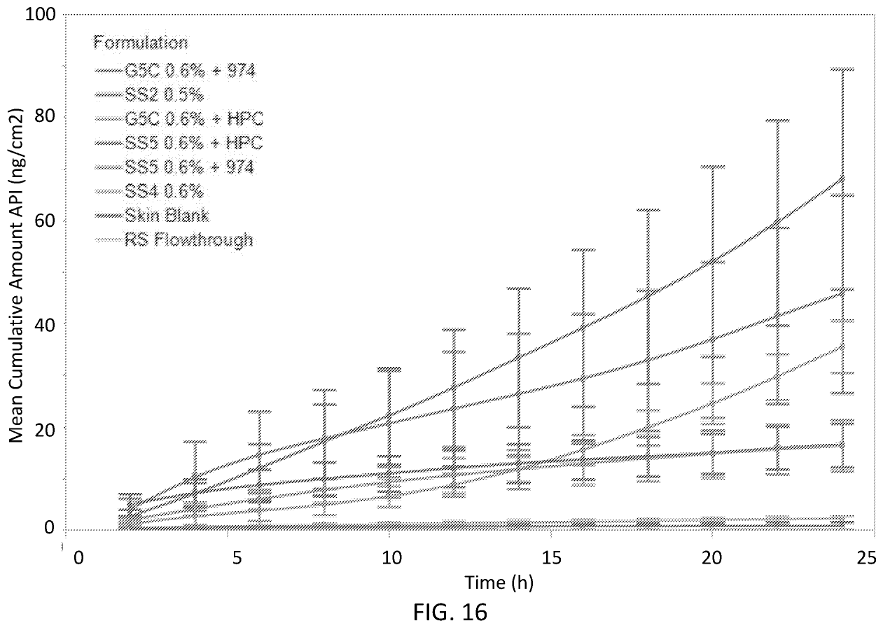

The mean cumulative amount (ng/cm²) of DMVT-503 permeated through the 1 cm² skin dosing area (i.e. drug detected in receptor solution) following application of six formulations (n=3-5 per formulation; 3 skin donors) was evaluated with out of trend results removed nonstatistically. The data in FIG. 16 is presented as mean±standard error of the mean using a receptor solution of citrate/phosphate buffer pH 5.6 with 0.01% Brij™-020.

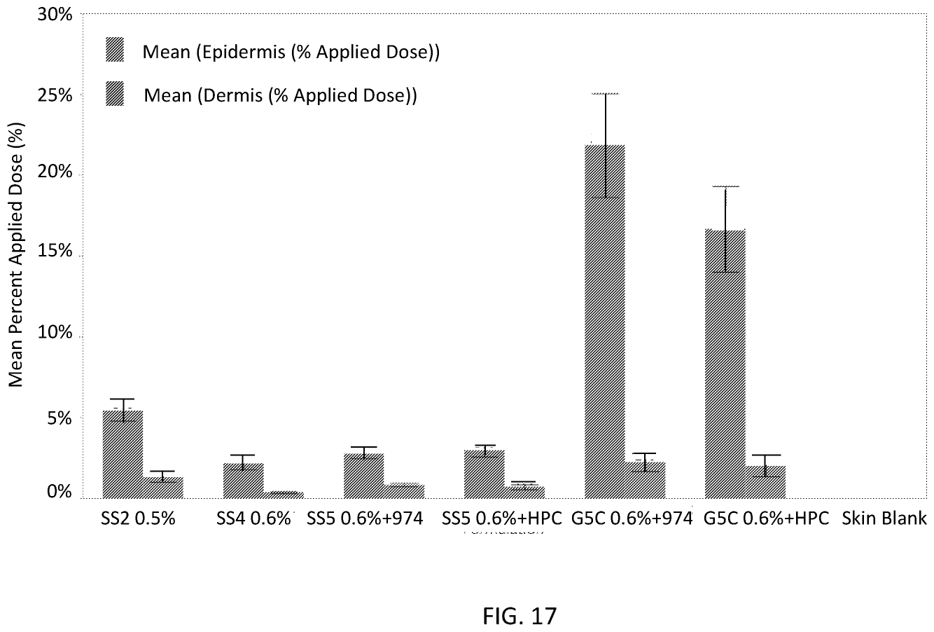

The mean cumulative amount of DMVT-503 (percent applied dose) recovered from epidermis and dermis 24 h post-application of six formulations was assessed. G5C 0.6% with 974 and G5C 0.6% with HPC delivered approximately 4-10-fold more DMVT-503 (p≤0.05) to the epidermis compared to the other four formulations. SS2 0.5% also delivered ca. 2-fold greater DMVT-503 to the epidermis than SS4 0.6% (p≤0.05). With respect to the dermis, G5C 0.6% with Carbopol 974 and G5C 0.6% with HPC delivered 5.6-6.8-fold more DMVT-503 compared to SS4 0.6% (p≤0.05). Each bar in FIG. 17 represents the mean (n=4-5 per formulation; 3 skin donors); error bars represent standard error of the mean. Table 9 provides the data as shown in the graph of FIG. 17.

TABLE 9
Mean Percent Applied Dose

| Formulation | Epidermis (%) | Dermis (%) |
|---|---|---|
| SS2 0.5% | 5 | 1 |
| SS4 0.6% | 2 | 0 |
| SS5 0.6% + 974 | 3 | 1 |
| SS5 0.6% + HPC | 3 | 1 |
| G5C 0.6% + 974 | 22 | 2 |
| G5C 0.6% + HPC | 17 | 2 |
| Blank | 0 | 0 |

### Example 7: Toxicokinetic Assessment of Systemic Exposure in Minipigs

Following Dermal Administration

Male and female göttingen minipigs were dosed daily for a period of 28 days in a GLP toxicity study with DMVT-503 gel at 0.1%, 0.3%, and 0.6%. DMVT-503 concentrations were quantitated using a validated LC-MS/MS method and toxicokinetic parameters were calculated for all dosing groups, sexes, and sampling occasions. The systemic exposures observed in this study are supportive of the effective delivery of DMVT-503 into and through minipig skin by the gel formulations tested. Table 10 presents the summary as mean±SD on Days 1 and 28 following daily dermal application of DMVT-503 gel formulations at 0.1%, 0.3%, and 0.6% (2, 6, and 12 mg/kg/day, respectively).

TABLE 10
DMVT-503 Toxieokinetie Parameters in Male
and Female Göttingen Minipig Plasma

| | | | Dose | Dose | | | |
|---|---|---|---|---|---|---|---|
| | | | (mg/ | Concen- | Cₘₐₓ | AUC₀₋₂₄^{a} | |
| | | | kg/ | tration | (ng/ | (ng * | Tₘₐₓ^{b} |
| Analyte | Day | Sex | day) | (%) | mL) | hr/mL) | (hr) |
| DMVT- | 1 | Male | 2 | 0.1 | 35.1 | 307 | 24 (24-24) |
| 503 | | | 6 | 0.3 | 53.0 | 478 | 24 (24-24) |
| | | | 12 | 0.6 | 103 | 10500 | 24 (24-24) |
| | | Fe- | 2 | 0.1 | 37.3 | 323 | 24 (24-24) |
| | | male | 6 | 0.3 | 210 | 1750 | 24 (24-24) |
| | | | 12 | 0.6 | 171 | 1490 | 24 (24-24) |
| | 28 | Male | 2 | 0.1 | 19.6 | 3970 | 0 (0-1) |
| | | | 6 | 0.3 | 940 | 17500 | 2 (0-6) |
| | | | 12 | 0.6 | 1160 | 22300 | 1 (0-24) |
| | | Fe- | 2 | 0.1 | 193 | 3940 | 1 (0-24) |
| | | male | 6 | 0.3 | 487 | 8660 | 0 (0-1) |
| | | | 12 | 0.6 | 1370 | 25400 | 0 (0-2) |

^{a}AUC₀₋₂₄ is equal to AUC₀₋ₜ
^{b}Median (Min-Max)

1. A topical composition comprising about 0.1% to about 1.0% 2-(4-(4-(6-carbamoyl-3,5-dimethylpyrazin-2-yl)phenyl)cyclohexyl)acetic acid by weight of the topical composition, about 10.0% to about 50.0% propylene glycol by weight of the topical composition, about 30.0% to about 75.0% 2-(2-ethoxyethoxy)ethanol by weight of the topical composition, about 15.0% to about 50.0% PEG400 by weight of the topical composition, about 0.01% to about 5.0% butylated hydroxytoluene by weight of the topical composition, and optionally one or more pharmaceutically acceptable topical excipients.

2. The topical composition of claim 1, wherein the topical composition further comprises glycerin, cellulose polymers, Carbomer Homopolymer Type A, Carbomer Homopolymer Type B, Carbomer Homopolymer Type C, or combinations thereof.

3-9. (canceled)

10. The topical composition of claim 2, wherein the glycerin is at a concentration from about 5.0% to about 25.0% by weight of the topical composition.

11. The topical composition of claim 2, wherein the cellulose polymer is at a concentration from about 0.1% to about 5.0% by weight of the topical composition.

12. The topical composition of claim 2, wherein the Carbomer Homopolymer Type A, Carbomer Homopolymer Type B, or Carbomer Homopolymer Type C is at a concentration from about 0.1% to about 5.0% by weight of the topical composition.

13. A topical composition comprising about 0.1% to about 1.0% 2-(4-(4-(6-carbamoyl-3,5-dimethylpyrazin-2-yl)phenyl)cyclohexyl)acetic acid by weight of the topical composition, about 30.0% to about 75.0% 2-(2-ethoxyethoxy)ethanol by weight of the topical composition, about 15.0% to about 65.0% PEG400 by weight of the topical composition, about 5.0% to about 25.0% glycerin by weight of the topical composition, about 0.1% to about 5.0% cellulose polymer(s) by weight of the topical composition, about 0.01% to about 5.0% butylated hydroxy toluene by weight of the topical composition, and optionally one or more pharmaceutically acceptable topical excipients.

14-19. (canceled)

20. A topical composition comprising about 0.1% to about 1.0% 2-(4-(4-(6-carbamoyl-3,5-dimethylpyrazin-2-yl)phenyl)cyclohexyl)acetic acid by weight of the topical composition, about 20.0% to about 50.0% propylene glycol by weight of the topical composition, about 25.0% to about 65.0% 2-(2-ethoxyethoxy)ethanol by weight of the topical composition, about 5.0% to about 25.0% glycerin by weight of the topical composition, about 50.0% to about 25.0% dimethyl isosorbide by weight of the topical composition, about 0.01% to about 5.0% butylated hydroxy toluene by weight of the topical composition, and optionally one or more pharmaceutically acceptable topical excipients.

21. The topical composition of claim 20, wherein the topical composition further comprises cellulose polymers, Carbomer Homopolymer Type A, Carbomer Homopolymer Type B, Carbomer Homopolymer Type C, or combinations thereof.

22-28. (canceled)

29. The topical composition of claim 21, wherein the cellulose polymer is at a concentration from about 0.1% to about 5.0% by weight of the topical composition.

30. The topical composition of claim 21, wherein the Carbomer Homopolymer Type A, Carbomer Homopolymer Type B, or Carbomer Homopolymer Type C is at a concentration from about 0.1% to about 5.0% by weight of the topical composition.

31. A topical composition comprising about 0.1% to about 1.0% 2-(4-(4-(6-carbamoyl-3,5-dimethylpyrazin-2-yl)phenyl)cyclohexyl)acetic acid by weight of the topical composition, about 0.5% to about 5.0% benzyl alcohol by weight of the topical composition, about 1.0% to about 10.0% PEG 400 by weight of the topical composition, about 1.0% to about 20.0% diisopropyl adipate by weight of the topical composition, about 1.0% to about 20.0% diethyl sebacate by weight of the topical composition, about 1.0% to about 20.0% dimethyl isosorbide by weight of the topical composition, about 1.0% to about 30.0% 2-(2-ethoxyethoxy)ethanol by weight of the topical composition, about 1.0% to about 15.0% hexylene glycol by weight of the topical composition, about 1.0% to about 10.0% polysorbate 80 by weight of the topical composition, and optionally one or more pharmaceutically acceptable topical excipients.

32-40. (canceled)

41. A topical composition comprising about 0.1% to about 1.0% 2-(4-(4-(6-carbamoyl-3,5-dimethylpyrazin-2-yl)phenyl)cyclohexyl)acetic acid by weight of the topical composition, about 0.5% to about 5.0% benzyl alcohol by weight of the topical composition, about 30.0% to about 60.0% PEG 400 by weight of the topical composition, about 1.0% to about 30.0% 2-(2-ethoxyethoxy)ethanol by weight of the topical composition, about 1.0% to about 10.0% polysorbate 80 by weight of the topical composition, and optionally one or more pharmaceutically acceptable topical excipients.

42-46. (canceled)

47. The topical composition of claim 41, further comprising diisopropyl adipate in a concentration from about 1.0% to about 25.0% by weight of the topical composition.

48. The topical composition of claim 41, further comprising hexylene glycol in a concentration from about 1.0% to about 20.0% by weight of the topical composition.

49. A method of treating skin conditions in a patient in need thereof comprising topically applying a topical composition of claim 1.

50-59. (canceled)

60. The method of claim 49, wherein the skin condition is selected from the group consisting of inflammatory acne, non-inflammatory acne, acne, comedonal acne, inflammatory acne, papular acne, pustular acne, cystic acne, follicular hyperkeratinization, seborrheic cysts, seborrhea, sebostasis, seborrheic dermatitis, sebaceous adenomas, sebaceous hyperplasia, and excess sebum production, dermatitis, atopic dermatitis, seborrheic dermatitis, alopecia, contact dermatitis, psoriasis, urticaria eczema, burns, sunburn, pancreatitis, hepatitis, lichen planus, scleritis, scleroderma, dermatomyositis, itching associated with any of the preceding conditions, and any combination thereof.

61. A method of treating skin conditions in a patient in need thereof comprising topically applying a topical composition of claim 13.

62-66. (canceled)

67. The method of claim 61, wherein the skin condition is selected from the group consisting of inflammatory acne, non-inflammatory acne, acne, comedonal acne, inflammatory acne, papular acne, pustular acne, cystic acne, follicular hyperkeratinization, seborrheic cysts, seborrhea, sebostasis, seborrheic dermatitis, sebaceous adenomas, sebaceous hyperplasia, and excess sebum production, dermatitis, atopic dermatitis, seborrheic dermatitis, alopecia, contact dermatitis, psoriasis, urticaria eczema, burns, sunburn, pancreatitis, hepatitis, lichen planus, scleritis, scleroderma, dermatomyositis, itching associated with any of the preceding conditions, and any combination thereof.

68. A method of treating skin conditions in a patient in need thereof comprising topically applying a topical composition of claim 20.

69-77. (canceled)

78. The method of claim 68, wherein the skin condition is selected from the group consisting of inflammatory acne, non-inflammatory acne, acne, comedonal acne, inflammatory acne, papular acne, pustular acne, cystic acne, follicular hyperkeratinization, seborrheic cysts, seborrhea, sebostasis, seborrheic dermatitis, sebaceous adenomas, sebaceous hyperplasia, and excess sebum production, dermatitis, atopic dermatitis, seborrheic dermatitis, alopecia, contact dermatitis, psoriasis, urticaria eczema, burns, sunburn, pancreatitis, hepatitis, lichen planus, scleritis, scleroderma, dermatomyositis, itching associated with any of the preceding conditions, and any combination thereof.

79. A method of treating skin conditions in a patient in need thereof comprising topically applying a topical composition of claim 31.

80-88. (canceled)

89. The method of claim 79, wherein the skin condition is selected from the group consisting of inflammatory acne, non-inflammatory acne, acne, comedonal acne, inflammatory acne, papular acne, pustular acne, cystic acne, follicular hyperkeratinization, seborrheic cysts, seborrhea, sebostasis, seborrheic dermatitis, sebaceous adenomas, sebaceous hyperplasia, and excess sebum production, dermatitis, atopic dermatitis, seborrheic dermatitis, alopecia, contact dermatitis, psoriasis, urticaria eczema, burns, sunburn, pancreatitis, hepatitis, lichen planus, scleritis, scleroderma, dermatomyositis, itching associated with any of the preceding conditions, and any combination thereof.

90. A method of treating skin conditions in a patient in need thereof comprising topically applying a topical composition of claim 41.

91-97. (canceled)

98. The method of claim 90, wherein the skin condition is selected from the group consisting of inflammatory acne, non-inflammatory acne, acne, comedonal acne, inflammatory acne, papular acne, pustular acne, cystic acne, follicular hyperkeratinization, seborrheic cysts, seborrhea, sebostasis, seborrheic dermatitis, sebaceous adenomas, sebaceous hyperplasia, and excess sebum production, dermatitis, atopic dermatitis, seborrheic dermatitis, alopecia, contact dermatitis, psoriasis, urticaria eczema, burns, sunburn, pancreatitis, hepatitis, lichen planus, scleritis, scleroderma, dermatomyositis, itching associated with any of the preceding conditions, and any combination thereof.
